(19) United States
(12) Patent Application Publication
(10) Pub. No.: US 2011/0167518 A1
(43) Pub. Date: Jul. 7, 2011

(54) ZEA MAYS SARK PROMOTER AND USES THEREOF

(75) Inventor: Norbert Brugière, Johnston, IA (US)

(73) Assignee: PIONEER HI-BRED INTERNATIONAL, INC., Johnston, IA (US)

(21) Appl. No.: 12/985,504

(22) Filed: Jan. 6, 2011

Related U.S. Application Data
(60) Provisional application No. 61/292,564, filed on Jan. 6, 2010.

(51) Int. Cl. A01H 5/00; C07H 21/04; A01H 5/10; C12N 15/82; C12N 5/10

(52) U.S. Cl. 800/279; 536/24.1; 800/298; 800/320.1; 800/320; 800/320.3; 800/320.2; 800/301; 800/302; 800/278; 800/284; 800/281; 800/287; 435/320.1; 435/419; 435/412; 435/468

(57) ABSTRACT

The present invention provides compositions and methods for regulating expression of heterologous nucleotide sequences in a plant. Compositions are novel nucleotide sequences for a Zea maize SARK promoter isolated from maize. A method for expressing a heterologous nucleotide sequence in a plant using the regulatory sequences disclosed herein is provided. The method comprises transforming a plant cell to comprise a heterologous nucleotide sequence operably linked to one or more of the regulatory sequences of the present invention and regenerating a stably transformed plant from the transformed plant cell.

### CROSS REFERENCE

This utility application claims the benefit U.S. Provisional Application No. 61/292,564, filed Jan. 6, 2010, which is incorporated herein by reference.

### FIELD OF THE INVENTION

The present invention relates to the field of plant molecular biology, more particularly to regulation of gene expression in plants.

### BACKGROUND OF THE INVENTION

Expression of heterologous DNA sequences in a plant host is dependent upon the presence of operably linked regulatory elements that are functional within the plant host. Choice of the regulatory element will determine when and where within the organism the heterologous DNA sequence is expressed. Where continuous expression is desired throughout the cells of a plant and/or throughout development, constitutive promoters are utilized. In contrast, where gene expression in response to a stimulus is desired, inducible promoters are the regulatory element of choice. Where expression in specific tissues or organs are desired, tissue-specific promoters may be used. That is, they may drive expression in specific tissues or organs. Such tissue-specific promoters may be temporally constitutive or inducible. In either case, additional regulatory sequences upstream and/or downstream from a core promoter sequence may be included in expression constructs of transformation vectors to bring about varying levels of expression of heterologous nucleotide sequences in a transgenic plant. See, for example, U.S. Pat. No. 5,850,018.

As more genes become accessible, a greater need exists for transformed plants with multiple genes. These multiple exogenous genes typically need to be controlled by separate regulatory sequences. Further, some genes should be regulated constitutively whereas other genes should be expressed at certain developmental stages or locations in the transgenic organism. Accordingly, a variety of regulatory sequences having diverse effects is needed.

Diverse regulatory sequences are also needed as undesirable biochemical interactions can result from using the same regulatory sequence to control more than one gene. For example, transformation with multiple copies of a regulatory element may cause problems, such that expression of one or more genes may be affected.

Regulatory sequences may also be useful in controlling temporal and/or spatial expression of endogenous DNA. For example, specialized tissues are involved in fertilization and seed development. Identification of promoters which are active in these seed tissues is of interest.

In grain crops of agronomic importance, seed formation is the ultimate goal of plant development. Seeds are harvested for use in food, feed and industrial products. The quantities and proportions of protein, oil and starch components in those seeds determine their utility and value.

The timing of seed development is critical. Environmental conditions at any point prior to fertilization through seed maturation may affect the quality and quantity of seed produced. In particular, the first 10 to 12 days after pollination (the lag phase) are critical in maize seed development. Several developmental events during the lag phase are important determinants of the fate of subsequent seed growth and development. (Cheikh, et al., (1994) Plant Physiology 106:45-51). Therefore, a means to influence plants response to stress during this phase of growth, is of interest. Identification of a promoter sequence active in leaves before the onset of senescence and driving a gene conferring tolerance to the stress would therefore be useful.

Specialized plant tissues are central to seed development. Following fertilization, developing seeds become sinks for carbon translocated via the phloem from sites of photosynthesis. However, developing cereal seeds have no direct vascular connections with the plant; instead, a short-distance transport mechanism operates to move the assimilates from the vascular tissues to the endosperm and embryo. For example, in maize, photosynthate enters the seed via the pedicel; in wheat, via the nuclear projection and the aleurone layer. It is possible that this short-distance assimilate pathway between the phloem and the endosperm can operate to regulate the rate of sucrose transport into the grain. (Bewley and Black, Seeds: Physiology of Development and Germination New York, Plenum Press, 1985 pp. 38-39). A promoter capable of driving expression of a gene that would maintain source strength under drought stress would therefore allow for a better flow of assimilates to developing kernels and would result in improved yield under unfavorable conditions.

Water stress to the plant around anthesis and during grain filling often results in seed abortion or restricted development. Studies suggest that sucrose continues to unload from the phloem at low ovary water potential, but it accumulates in the symplasm and apoplasm of the pedicel because of low invertase activity. (Zinselmeier, et al., (1995) Plant Physiol. 107:385-391). This conclusion is supported by the findings of Miller and Chourey (Plant Cell 4:297-305 (1992)), who showed that developmental failure of miniature-1 seeds of maize was linked to lack of invertase activity in the pedicel tissue during the early stages of seed development.

To achieve yield stability in high-density plantings, under drought conditions or in other adverse environments, modification of carbohydrate metabolism during early ear and kernel development may be desirable. Effective control of genes involved in carbohydrate metabolism is dependent on identification and use of a promoter with high levels of tissue and temporal specificity. In light of the important contributions of leaf photosynthetic potential to kernel growth under drought stress identification of a promoter sequence capable of driving gene expression at the beginning of leaf senescence would be desirable.

Maize cytokinins are members of a class of plant hormones important in the control of cell division and in regulation of plant growth and structure. Elevated cytokinin levels are associated with the development of seeds in higher plants; exogenous leaf cytokinin application has been shown to directly correlate with increased kernel yield in maize.

The invention disclosed herein is a maize promoter called ZM-SARK (Senescence Activated Receptor Kinase) PRO. It was identified as the promoter of a gene which expression is induced during right before the onset of senescence g based on homology of the protein encoded by the ZM-SARK gene with the protein sequence of a Senescence Activated Receptor Kinase (SARK) gene from Phaseolus vulgaris (see, Hajouj, et al., (2001) Plant Physiology 126(3):1341-1342). The ZM-SARK gene coding sequence (FIG. 2a) was identified from our proprietary cDNA collection and was found to encode a protein of 939 amino acid with similarity to Phaseolus vulgaris SARK protein (43.2% similarity and 30.8% identity at the amino acid level) (FIG. 1). The promoter sequence of the corresponding gene (FIG. 2b) was identified based on proprietary gene modeling tool and cloned from B73 genomic DNA using gene specific primers (Zm-SARK genomic sequence is provided as SEQ ID NO: 3). Preliminary analysis of ZM-SARK expression by Northern blot in ear-leaves harvested at different time after flowering are consistent with higher expression of ZM-SARK just prior to the onset of leaf senescence, immediately prior to the loss of chlorophyll (FIG. 3). In leaves, ZM-SARK expression was shown to follow a circadian expression where expression decreased during the day and increased at night (FIG. 4) (Hayes, et al., (2010) PLoS ONE 5:9). Lynx MPSS profiling indicates that the promoter is also driving expression in roots (FIG. 5). Functional evaluation of ZM-SARK PRO is currently studied using a glucuronidase (GUS) reporter gene. Preliminary results indicate that the ZM-SARK PRO sequence described therein is sufficient to drive expression of the reporter gene in root, indicating that the sequence can function as a promoter in planta. Results presented in FIG. 6 show GUS activity in roots of maize germinations transformed by bombardment with a plasmid containing a ZM-SARK PRO: GUS construct.

The ZM-SARK promoter could be used in combination with a cytokinin biosynthetic gene (ZM-IPT2, for example) to reduce drought induced leaf senescence in order to improve kernel fill and yield performance under water limiting conditions. Recent data indicate that expression of IPT under the control of the Phaseolus SARK promoter can increased the stability of photosynthetic proteins (Rivero, et al., (2010) Plant and Cell Physiology 51:1929-1941).

A novel and heretofore undescribed utility of the maize ZM-SARK promoter presented here is using a maize promoter in combination with a native maize biosynthetic gene to increase cytokinin levels and delay drought induced leaf senescence. Utilization of ZM-SARK promoter would overcome the problem and disadvantages of using dicot and bacterial genetic components in maize. For example, a dicot promoter would not likely function as well in monocot. Similarly a maize cytokinin biosynthetic enzyme could be more efficacious than a bacterial enzyme.

A full-length promoter sequence of the isolated maize SARK promoter and functional fragments and variants thereof and the use of such sequences with heterologous nucleotide sequences of interest, are described in the present invention. Unless otherwise specified, the notation “ZM-SARK PRO” in reference to the subject promoter includes SEQ ID NO: 1 and any functional fragments or variants thereof.

### SUMMARY OF THE INVENTION

The invention is to a regulatory element that regulates transcription in order to delay drought induced leaf senescence.

It is an object of the present invention to provide a novel nucleotide sequence for modulating gene expression in a plant.

It is a further object of the present invention to provide an isolated promoter capable of driving transcription in a tissue-preferred manner.

It is a further object of the present invention to provide an isolated promoter sequence which functions with native maize genes to increase cytokinin levels.

It is a further object of the present invention to provide a method of improved control of an endogenous or exogenous product in a transformed plant.

It is a further object of the present invention to provide a method for effecting useful changes in the phenotype of a transformed plant.

It is a further object of the present invention to provide a method for producing a novel product in a transformed plant.

It is a further object of the present invention to provide a method for producing a novel function in a transformed plant.

It is a further object of the present invention to provide a method for modulating the timing or rate of leaf senescence of a transformed plant.

It is a further object of the present invention to provide a method for regulating the level of cytokinin within a plant.

Therefore, in one aspect, the present invention relates to an isolated nucleic acid comprising a member selected from the group consisting of:
a) nucleic acids capable of driving expression in at or just prior the onset of senescence in a circadian regulated manner as well as in the root of maize germination root tissue;
b) nucleic acids comprising a functional variant or fragment of at least 20 contiguous nucleotides of the sequence set forth in SEQ ID NO: 1;
c) the nucleic acid sequence of SEQ ID NO:1; and
d) nucleic acids that hybridize to any one of a), b) or c) under stringent conditions, wherein stringent conditions include hybridization at 42° C. in a solution of 50% (w/v) formamide, 6×SSC, 0.5% SDS, 100 ug/ml salmon sperm, washed with 0.5% SDS and 0.1×SSC at about 65° C. for 30 minutes and repeated.

In other aspects, the present invention relates to expression cassettes comprising the promoter operably linked to a nucleotide sequence, vectors containing said expression cassette and plants stably transformed with at least one said expression cassette.

In a further aspect, the present invention relates to a method for modulating expression in the seed, root, stalk or vascular tissue of a stably transformed plant comprising the steps of (a) transforming a plant cell with an expression cassette comprising the promoter of the present invention operably linked to at least one nucleotide sequence; (b) growing the plant cell under plant growing conditions and (c) regenerating a stably transformed plant from the plant cell wherein said linked nucleotide sequence is expressed in the seed, root, stalk or vascular tissue.

Further embodiments are to expression cassettes, transformation vectors, plants, plant cells and plant parts comprising the above nucleotide sequences. The invention is further to methods of using the sequence in plants and plant cells. An embodiment of the invention further comprises the nucleotide sequences described above comprising a detectable marker.

### BRIEF DESCRIPTION OF THE FIGURES

FIG. 1. Alignment of SARK sequences (FIGS. 1a, b and c). The ZM-SARK gene was found to encode a protein (SEQ ID NO: 5) of 939 amino acid with similarity to Phaseolus vulgaris SARK protein (SEQ ID NO: 6) (43.2% similarity and 30.8% identity at the amino acid level), consensus is SEQ ID NO: 7.

FIG. 2. The ZM-SARK gene coding sequence (FIG. 2a) was identified from our proprietary cDNA collection. The promoter sequence of the corresponding gene (FIG. 2b) was identified based on proprietary gene modeling tool and cloned from B73 genomic DNA using gene specific primers.

FIG. 3. Preliminary analysis of ZM-SARK expression by Northern blot in ear-leaves harvested at different time after flowering are consistent with higher expression of ZM-SARK just prior to the onset of leaf senescence, immediately prior to the loss of chlorophyll.

FIG. 4. In leaves, ZM-SARK expression was shown to follow a circadian expression where expression decreased during the day and increased at night.

FIG. 5. Lynx MPSS profiling indicates that the promoter is also driving expression in roots.

FIG. 6. GUS activity in roots of maize germinations transformed by bombardment with a plasmid containing a ZM-SARK PRO: GUS construct. Dark stain indicates GUS activity detection.

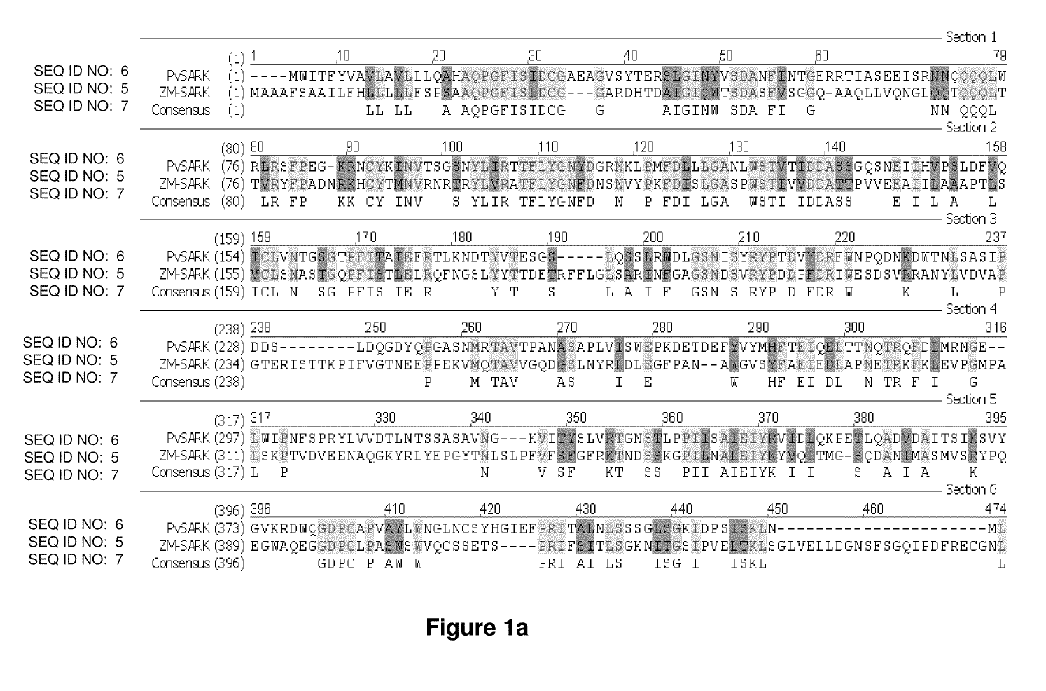

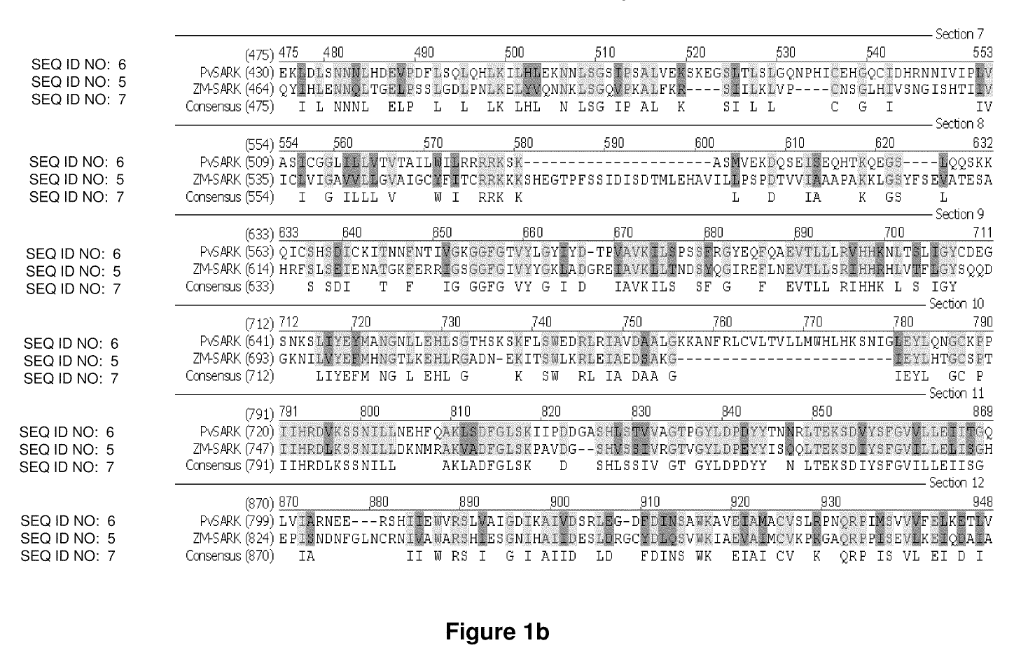

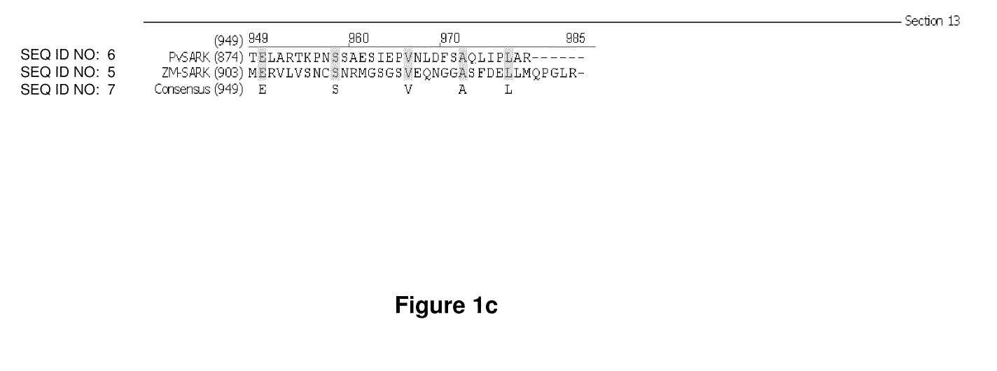

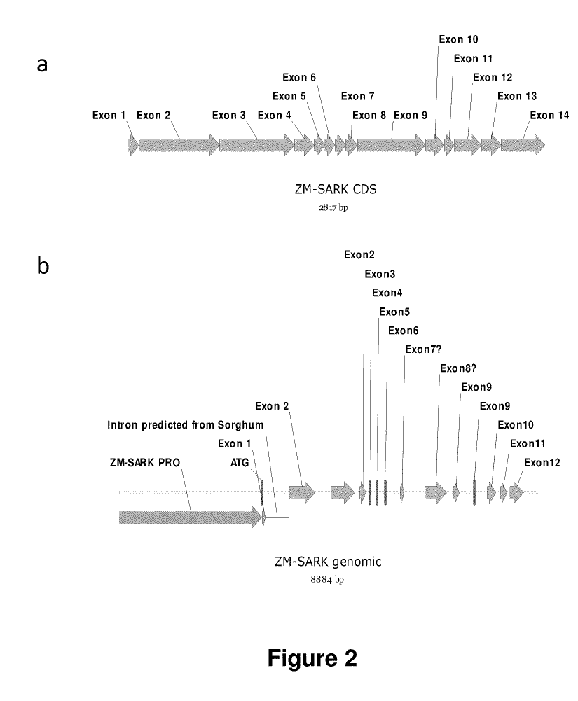

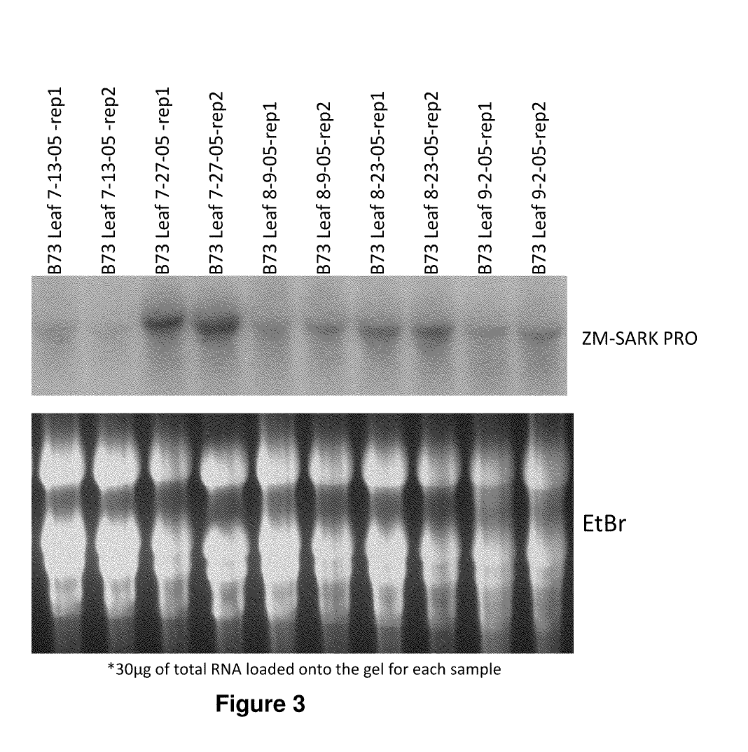

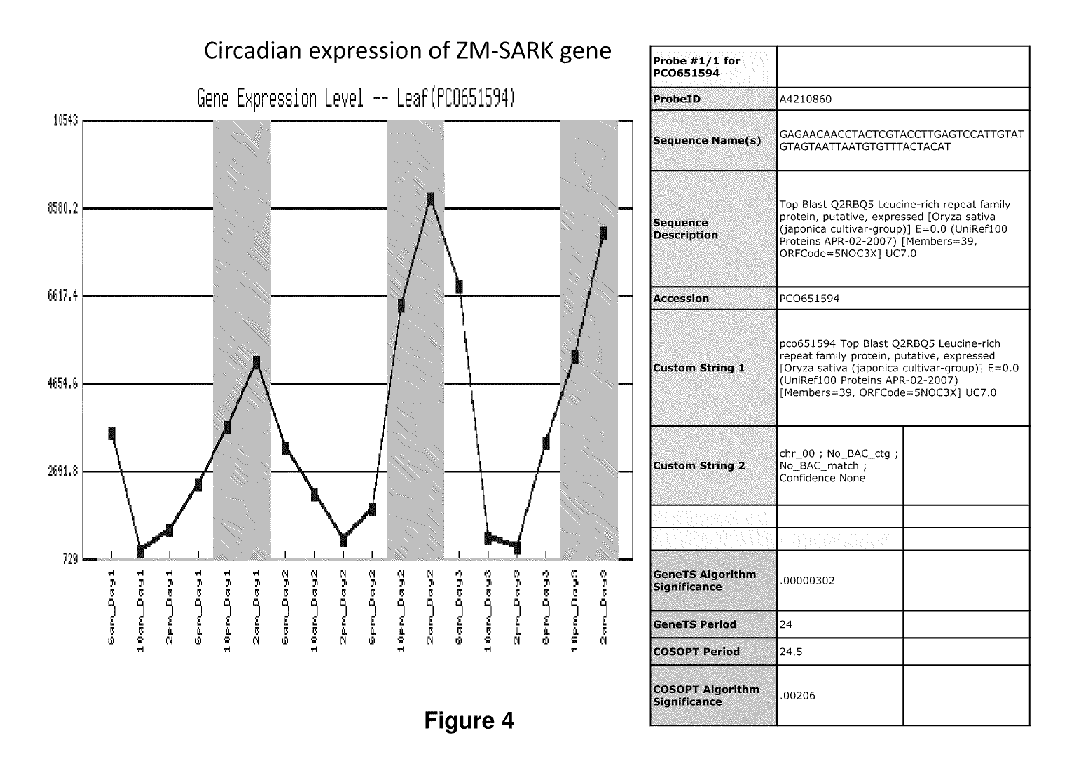

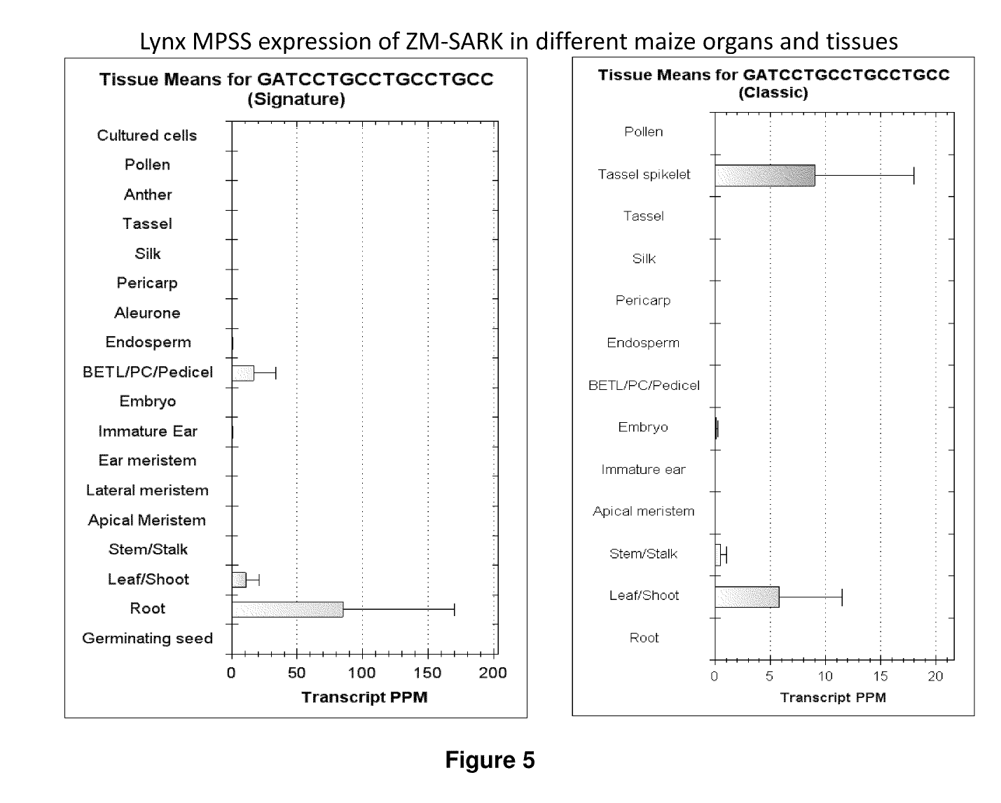

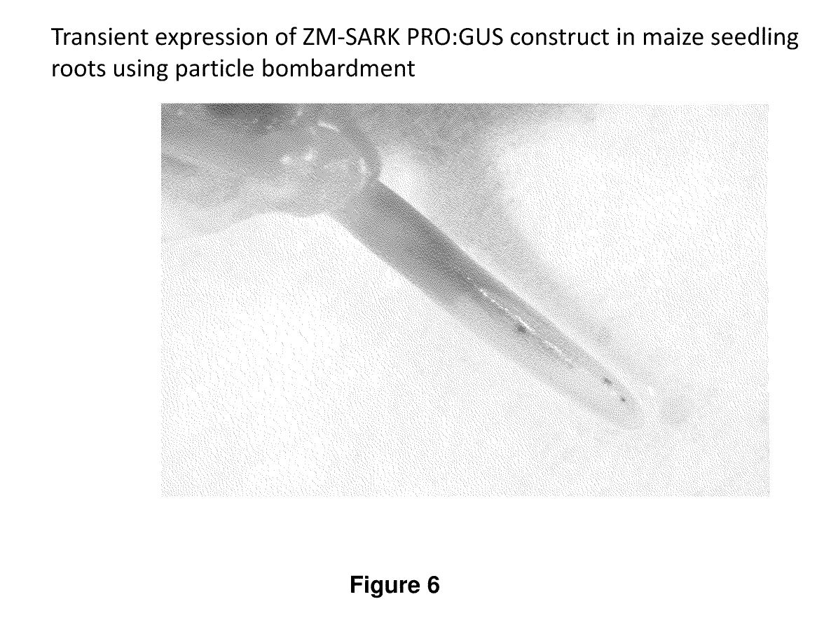

### DETAILED DESCRIPTION OF THE INVENTION

In accordance with the invention, a nucleotide sequence is provided that favors initiation of transcription in specific tissues, including roots at early stages of development and leaves just prior or at the onset of leaf senescence in a circadian regulated fashion. The sequence of the invention comprises transcriptional initiation regions associated with said tissues. Thus, the compositions of the present invention comprise a novel nucleotide sequence for a plant promoter.

A method for expressing an isolated nucleotide sequence in a plant using the regulatory sequences disclosed herein is provided. The method comprises transforming a plant cell with a transformation vector that comprises an isolated nucleotide sequence operably linked to one or more of the plant regulatory sequences of the present invention and regenerating a stably transformed plant from the transformed plant cell. In this manner, the regulatory sequences are useful for controlling the expression of endogenous as well as exogenous products in a root-preferred manner.

Frequently it is desirable to have preferential expression of a DNA sequence in a tissue of an organism. For example, increased resistance of a plant to insect attack might be accomplished by genetic manipulation of the plant's genome to comprise a tissue-specific promoter operably linked to a heterologous insecticide gene such that the insect-deterring substances are specifically expressed in the susceptible plant tissues. Preferential expression of the heterologous nucleotide sequence in the appropriate tissue reduces the drain on the plant's resources that occurs when a constitutive promoter initiates transcription of a heterologous nucleotide sequence throughout the cells of the plant.

Alternatively, it might be desirable to inhibit expression of a native DNA sequence within a plant's tissues to achieve a desired phenotype. In this case, such inhibition might be accomplished with transformation of the plant to comprise a tissue-specific promoter operably linked to an antisense nucleotide sequence, such that tissue-specific expression of the antisense sequence produces an RNA transcript that interferes with translation of the mRNA of the native DNA sequence in a subset of the plant's cells.

Under the regulation of the root-specific regulatory elements will be a sequence of interest, which will provide for modification of the phenotype of the root. Such modification includes modulating the production of an endogenous product, as to amount, relative distribution or the like or production of an exogenous expression product to provide for a novel function or product in the root.

### DEFINITIONS

By “root-preferred” is intended favored expression in the plant root, the root vasculature of a plant and the like.

By “regulatory element” is intended sequences responsible expression of the associated coding sequence including, but not limited to, promoters, terminators, enhancers, introns and the like.

By “terminator” is intended sequences that are needed for termination of transcription: a regulatory region of DNA that causes RNA polymerase to disassociate from DNA, causing termination of transcription.

By “promoter” is intended a regulatory region of DNA capable of regulating the transcription of a sequence linked thereto. It usually comprises a TATA box capable of directing RNA polymerase II to initiate RNA synthesis at the appropriate transcription initiation site for a particular coding sequence.

A promoter may additionally comprise other recognition sequences generally positioned upstream or 5′ to the TATA box, referred to as upstream promoter elements, which influence the transcription initiation rate and further include elements which impact spatial and temporal expression of the linked nucleotide sequence. It is recognized that having identified the nucleotide sequences for the promoter region disclosed herein, it is within the state of the art to isolate and identify further regulatory elements in the 5′ region upstream from the particular promoter region identified herein. Thus the promoter region disclosed herein may comprise upstream regulatory elements such as those responsible for tissue and temporal expression of the coding sequence and may include enhancers, the DNA response element for a transcriptional regulatory protein, ribosomal binding sites, transcriptional start and stop sequences, translational start and stop sequences, activator sequence and the like.

In the same manner, the promoter elements which enable expression in the desired tissue such as the root can be identified, isolated and used with other core promoters to confirm root-preferred expression. By core promoter is meant the minimal sequence required to initiate transcription, such as the sequence called the TATA box which is common to promoters in genes encoding proteins. Thus the upstream promoter of SARK can optionally be used in conjunction with its own or core promoters from other sources. The promoter may be native or non-native to the cell in which it is found.

The isolated promoter sequence of the present invention can be modified to provide for a range of expression levels of the isolated nucleotide sequence. Less than the entire promoter region can be utilized and the ability to drive root-preferred expression retained. It is recognized that expression levels of mRNA can be modulated with specific deletions of portions of the promoter sequence. Thus, the promoter can be modified to be a weak or strong promoter. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level or at about 1/10 transcripts to about 1/100 transcripts to about 1/1,000 transcripts. Generally, at least about 20 nucleotides of an isolated promoter sequence will be used to drive expression of a nucleotide sequence.

It is recognized that to increase transcription levels enhancers can be utilized in combination with the promoter regions of the invention. Enhancers are nucleotide sequences that act to increase the expression of a promoter region. Enhancers are known in the art and include the SV40 enhancer region, the 35S enhancer element and the like.

The promoter of the present invention can be isolated from the 5′ region of its native coding region or 5′ untranslated region (5′ UTR). Likewise the terminator can be isolated from the 3′ region flanking its respective stop codon. The term “isolated” refers to material, such as a nucleic acid or protein, which is: (1) substantially or essentially free from components which normally accompany or interact with the material as found in its naturally occurring environment or (2) if the material is in its natural environment, the material has been altered by deliberate human intervention to a composition and/or placed at a locus in a cell other than the locus native to the material. Methods for isolation of promoter regions are well known in the art.

The promoter regions of the invention may be isolated from any plant, including, but not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), millet (Panicum spp.), potato (Solanum tuberosum), peanuts (Arachis hypogaea), cotton (Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), oats (Avena sativa), barley (Hordeum vulgare), vegetables, ornamentals, and conifers. Preferably, plants include corn, soybean, sunflower, safflower, canola, wheat, barley, rye, alfalfa and sorghum.

Promoter sequences from other plants may be isolated according to well-known techniques based on their sequence homology to the homologous coding region of the coding sequences set forth herein. In these techniques, all or part of the known coding sequence is used as a probe which selectively hybridizes to other sequences present in a population of cloned genomic DNA fragments (i.e., genomic libraries) from a chosen organism. Methods are readily available in the art for the hybridization of nucleic acid sequences. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993) and Current Protocols in Molecular Biology, Chapter 2, Ausubel, et al., Eds., Greene Publishing and Wiley-Interscience, New York (1995).

“Functional variants” of the regulatory sequences are also encompassed by the compositions of the present invention. Functional variants include, for example, the native regulatory sequences of the invention having one or more nucleotide substitutions, deletions or insertions. Functional variants of the invention may be created by site-directed mutagenesis, induced mutation or may occur as allelic variants (polymorphisms).

As used herein, a “functional fragment” is a regulatory sequence variant formed by one or more deletions from a larger regulatory element. For example, the 5′ portion of a promoter up to the TATA box near the transcription start site can be deleted without abolishing promoter activity, as described by Opsahl-Sorteberg, et al., (2004) Gene 341:49-58. Such variants should retain promoter activity, particularly the ability to drive expression in root or root tissues. Activity can be measured by Northern blot analysis, reporter activity measurements when using transcriptional fusions, and the like. See, for example, Sambrook, et al., (1989) Molecular Cloning: A Laboratory Manual (2nd ed. Cold Spring Harbor Laboratory, Cold Spring Harbor, N.Y.), herein incorporated by reference.

Functional fragments can be obtained by use of restriction enzymes to cleave the naturally occurring regulatory element nucleotide sequences disclosed herein; by synthesizing a nucleotide sequence from the naturally occurring DNA sequence or can be obtained through the use of PCR technology. See particularly, Mullis, et al., (1987) Methods Enzymol. 155:335-350 and Erlich, ed. (1989) PCR Technology (Stockton Press, New York).

For example, a routine way to remove part of a DNA sequence is to use an exonuclease in combination with DNA amplification to produce unidirectional nested deletions of double stranded DNA clones. A commercial kit for this purpose is sold under the trade name Exo-Size™ (New England Biolabs, Beverly, Mass.). Briefly, this procedure entails incubating exonuclease III with DNA to progressively remove nucleotides in the 3′ to 5′ direction at 5′ overhangs, blunt ends or nicks in the DNA template. However, exonuclease III is unable to remove nucleotides at 3′, 4-base overhangs. Timed digests of a clone with this enzyme produces unidirectional nested deletions.

The entire promoter sequence or portions thereof can be used as a probe capable of specifically hybridizing to corresponding promoter sequences. To achieve specific hybridization under a variety of conditions, such probes include sequences that are unique and are preferably at least about 10 nucleotides in length and most preferably at least about 20 nucleotides in length. Such probes can be used to amplify corresponding promoter sequences from a chosen organism by the well-known process of polymerase chain reaction (PCR). This technique can be used to isolate additional promoter sequences from a desired organism or as a diagnostic assay to determine the presence of the promoter sequence in an organism. Examples include hybridization screening of plated DNA libraries (either plaques or colonies; see e.g., Innis, et al., (1990) PCR Protocols, A Guide to Methods and Applications, eds., Academic Press). By “seed” or “kernel” is intended to include the grain or ripened ovule of a plant, or more broadly, a propagative plant structure. The terms “seed” and “kernel” are used interchangeably herein.

By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. An endogenous promoter is operably linked to the endogenous coding region which it regulates.

In one typical embodiment, in the context of an over expression cassette, operably linked means that the nucleotide sequences being linked are contiguous and where necessary to join two or more protein coding regions contiguous and in the same reading frame. In the case where an expression cassette contains two or more protein coding regions joined in a contiguous manner in the same reading frame, the encoded polypeptide is herein defined as a “heterologous polypeptide” or a “chimeric polypeptide” or a “fusion polypeptide”. The cassette may additionally contain at least one additional coding sequence to be co-transformed into the organism. Alternatively, the additional coding sequence(s) can be provided on multiple expression cassettes.

The regulatory elements of the invention can be operably linked to the isolated nucleotide sequence of interest in any of several ways known to one of skill in the art. The isolated nucleotide sequence of interest can be inserted into a site within the genome which is 3′ to the promoter of the invention using site specific integration as described in U.S. Pat. No. 6,187,994, herein incorporated in it's entirety by reference.

The regulatory elements of the invention can be operably linked in expression cassettes along with isolated nucleotide sequences of interest for expression in the desired plant, more particularly in the root of the plant. Such an expression cassette is provided with a plurality of restriction sites for insertion of the nucleotide sequence of interest under the transcriptional control of the regulatory elements.

The isolated nucleotides of interest expressed by the regulatory elements of the invention can be used for directing expression of a sequence in plant tissues. This can be achieved by increasing expression of endogenous or exogenous products in root. Alternatively, the results can be achieved by providing for a reduction of expression of one or more endogenous products, particularly enzymes or cofactors in the root. This down regulation can be achieved through many different approaches known to one skilled in the art, including antisense, cosupression, use of hairpin formations or others and discussed infra. Importation or exportation of a cofactor also allows for control of root composition. It is recognized that the regulatory elements may be used with their native or other coding sequences to increase or decrease expression of an operably linked sequence in the transformed plant or seed.

General categories of genes of interest for the purposes of the present invention include for example, those genes involved in information, such as zinc fingers, those involved in communication, such as kinases and those involved in housekeeping, such as heat shock proteins. More specific categories of transgenes include genes encoding important traits for agronomics, insect resistance, disease resistance, herbicide resistance and grain characteristics. Still other categories of transgenes include genes for inducing expression of exogenous products such as enzymes, cofactors and hormones from plants and other eukaryotes as well as prokaryotic organisms.

Modifications that affect grain traits include increasing the content of oleic acid or altering levels of saturated and unsaturated fatty acids. Likewise, the level of root proteins, particularly modified root proteins that improve the nutrient value of the root, can be increased. This is achieved by the expression of such proteins having enhanced amino acid content.

Increasing the levels of lysine and sulfur-containing amino acids may be desired as well as the modification of starch type and content in the seed. Hordothionin protein modifications are described in WO 9416078, filed Apr. 10, 1997; WO 9638562, filed Mar. 26, 1997; WO 9638563, filed Mar. 26, 1997 and U.S. Pat. No. 5,703,409, issued Dec. 30, 1997. Another example is lysine and/or sulfur-rich root protein encoded by the soybean 2S albumin described in WO 9735023, filed Mar. 20, 1996 and the chymotrypsin inhibitor from barley, Williamson, et al., (1987) Eur. J. Biochem. 165:99-106.

Agronomic traits in roots can be improved by altering expression of genes that: affect the response of root, plant or seed growth and development during environmental stress, Cheikh-N, et al., (1994) Plant Physiol. 106(1):45-51 and genes controlling carbohydrate metabolism to reduce kernel abortion in maize, Zinselmeier, et al., (1995) Plant Physiol. 107(2):385-391.

It is recognized that any gene of interest, including the native coding sequence, can be operably linked to the regulatory elements of the invention and expressed in the root.

By way of illustration, without intending to be limiting, are examples of the types of genes which can be used in connection with the regulatory sequences of the invention.

1. Transgenes that confer resistance to Insects or disease and that encode:

(A) Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example, Jones, et al., (1994) Science 266:789 (cloning of the tomato Cf-9 gene for resistance to Cladosporium fulvum); Martin, et al., (1993) Science 262:1432 (tomato Pto gene for resistance to Pseudomonas syringae pv. tomato encodes a protein kinase); Mindrinos, et al., (1994) Cell 78:1089 (Arabidopsis RSP2 gene for resistance to Pseudomonas syringae); McDowell and Woffenden, (2003) Trends Biotechnol. 21(4):178-83 and Toyoda, et al., (2002) Transgenic Res. 11(6):567-82. A plant resistant to a disease is one that is more resistant to a pathogen as compared to the wild type plant.

(B) A Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC Accession Numbers 40098, 67136, 31995 and 31998. Other examples of Bacillus thuringiensis transgenes being genetically engineered are given in the following patents and patent applications and hereby are incorporated by reference for this purpose: U.S. Pat. Nos. 5,188,960; 5,689,052; 5,880,275; WO 91/14778; WO 99/31248; WO 01/12731; WO 99/24581; WO 97/40162 and U.S. patent application Ser. Nos. 10/032,717; 10/414,637 and 10/606,320.

(C) An insect-specific hormone or pheromone such as an ecdysteroid and juvenile hormone, a variant thereof, a mimetic based thereon or an antagonist or agonist thereof. See, for example, the disclosure by Hammock, et al., (1990) Nature 344:458 of baculovirus expression of cloned juvenile hormone esterase, an inactivator of juvenile hormone.

(D) An insect-specific peptide which, upon expression, disrupts the physiology of the affected pest. For example, see the disclosures of Regan, (1994) J. Biol. Chem. 269:9 (expression cloning yields DNA coding for insect diuretic hormone receptor); Pratt, et al., (1989) Biochem. Biophys. Res. Comm. 163:1243 (an allostatin is identified in Diploptera puntata); Chattopadhyay, et al., (2004) Critical Reviews in Microbiology 30(1):33-54 2004; Zjawiony, (2004) J Nat Prod 67(2):300-310; Carlini and Grossi-de-Sa, (2002) Toxicon, 40(11):1515-1539; Ussuf, et al., (2001) Curr Sci. 80(7):847-853 and Vasconcelos and Oliveira, (2004) Toxicon 44(4):385-403. See also, U.S. Pat. No. 5,266,317 to Tomalski, et al., who disclose genes encoding insect-specific toxins.

(E) An enzyme responsible for a hyperaccumulation of a monoterpene, a sesquiterpene, a steroid, hydroxycinnamic acid, a phenylpropanoid derivative or another non-protein molecule with insecticidal activity.

(F) An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See, PCT Application Number WO 93/02197 in the name of Scott, et al., which discloses the nucleotide sequence of a callase gene. DNA molecules which contain chitinase-encoding sequences can be obtained, for example, from the ATCC under Accession Numbers 39637 and 67152. See also, Kramer, et al., (1993) Insect Biochem. Molec. Biol. 23:691, who teach the nucleotide sequence of a cDNA encoding tobacco hookworm chitinase and Kawalleck, et al., (1993) Plant Molec. Biol. 21:673, who provide the nucleotide sequence of the parsley ubi4-2 polyubiquitin gene, U.S. patent application Ser. Nos. 10/389,432, 10/692,367 and U.S. Pat. No. 6,563,020.

(G) A molecule that stimulates signal transduction. For example, see the disclosure by Botella, et al., (1994) Plant Molec. Biol. 24:757 of nucleotide sequences for mung bean calmodulin cDNA clones and Griess, et al., (1994) Plant Physiol. 104:1467, who provide the nucleotide sequence of a maize calmodulin cDNA clone.

(H) A hydrophobic moment peptide. See, PCT Application Number WO 95/16776 and U.S. Pat. No. 5,580,852 (disclosure of peptide derivatives of Tachyplesin which inhibit fungal plant pathogens) and PCT Application Number WO 95/18855 and U.S. Pat. No. 5,607,914) (teaches synthetic antimicrobial peptides that confer disease resistance).

(I) A membrane permease, a channel former or a channel blocker. For example, see the disclosure by Jaynes, et al., (1993) Plant Sci. 89:43 of heterologous expression of a cecropin-beta lytic peptide analog to render transgenic tobacco plants resistant to Pseudomonas solanacearum.

(J) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See, Beachy, et al., (1990) Ann. Rev. Phytopathol. 28:451. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

(K) An insect-specific antibody or an immunotoxin derived therefrom. Thus, an antibody targeted to a critical metabolic function in the insect gut would inactivate an affected enzyme, killing the insect. Taylor, et al., Abstract #497, Seventh Int'l Symposium on Molecular Plant-microbe Interactions (Edinburgh, Scotland, 1994) (enzymatic inactivation in transgenic tobacco via production of single-chain antibody fragments).

(L) A virus-specific antibody. See, for example, Tavladoraki, et al., (1993) Nature 366:469, who show that transgenic plants expressing recombinant antibody genes are protected from virus attack.

(M) A developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See, Lamb, et al., (1992) Bio/Technology 10:1436. The cloning and characterization of a gene which encodes a bean endopolygalacturonase-inhibiting protein is described by Toubart, et al., (1992) Plant J. 2:367.

(N) A developmental-arrestive protein produced in nature by a plant. For example, Logemann, et al., (1992) Bio/Technology 10:305 have shown that transgenic plants expressing the barley ribosome-inactivating gene have an increased resistance to fungal disease.

(O) Genes involved in the Systemic Acquired Resistance (SAR) Response and/or the pathogenesis related genes. Briggs, (1995) Current Biology, 5(2):128-131, Pieterse and Van Loon, (2004) Curr. Opin. Plant Bio. 7(4):456-64 and Somssich, (2003) Cell 113(7):815-6.

(P) Antifungal genes (Cornelissen and Melchers, (1993) Pl. Physiol. 101:709-712 and Parijs, et al., (1991) Planta 183:258-264 and Bushnell, et al., (1998) Can. J. of Plant Path. 20(2):137-149. Also see, U.S. patent application Ser. No. 09/950,933.

(O) Detoxification genes, such as for fumonisin, beauvericin, moniliformin and zearalenone and their structurally related derivatives. For example, see, U.S. Pat. No. 5,792,931.

(R) Cystatin and cysteine proteinase inhibitors. See, U.S. patent application Ser. No. 10/947,979.

(S) Defensin genes. See, WO 03/000863 and U.S. patent application Ser. No. 10/178,213.

(T) Genes conferring resistance to nematodes. See, WO 03/033651 and Urwin, et al., (1998) Planta 204:472-479, Williamson (1999) Curr Opin Plant Bio. 2(4):327-31.

(U) Genes that confer resistance to Phytophthora Root Rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7 and other Rps genes. See, for example, Shoemaker, et al., Phytophthora Root Rot Resistance Gene Mapping in Soybean, Plant Genome IV Conference, San Diego, Calif. (1995).

(V) Genes that confer resistance to Brown Stem Rot, such as described in U.S. Pat. No. 5,689,035.

2. Transgenes that confer resistance to a herbicide such as:

(A) An herbicide that inhibits the growing point or meristem, such as an imidazolinone or a sulfonylurea. Exemplary genes in this category code for mutant ALS and AHAS enzyme as described, for example, by Lee, et al., (1988) EMBO J. 7:1241 and Miki, et al., (1990) Theor. Appl. Genet. 80:449, respectively. See also, U.S. Pat. Nos. 5,605,011; 5,013,659; 5,141,870; 5,767,361; 5,731,180; 5,304,732; 4,761,373; 5,331,107; 5,928,937 and 5,378,824 and International Publication Number WO 96/33270.

(B) Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes) and pyridinoxy or phenoxy proprionic acids and cycloshexones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587; 6,338,961; 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and International Publication Numbers EP1173580; WO 01/66704; EP1173581 and EP1173582. Glyphosate resistance is also imparted to plants that express a gene that encodes a glyphosate oxido-reductase enzyme as described more fully in U.S. Pat. Nos. 5,776,760 and 5,463,175. In addition glyphosate resistance can be imparted to plants by the over expression of genes encoding glyphosate N-acetyltransferase. See, for example, PCT Number US 01/46227; U.S. patent application Ser. Nos. 10/427,692 and 10/427,692. A DNA molecule encoding a mutant aroA gene can be obtained under ATCC Accession Number 39256 and the nucleotide sequence of the mutant gene is disclosed in U.S. Pat. No. 4,769,061 to Comai. EP Patent Application Number 0 333 033 to Kumada, et al., and U.S. Pat. No. 4,975,374 to Goodman, et al., disclose nucleotide sequences of glutamine synthetase genes which confer resistance to herbicides such as L-phosphinothricin. The nucleotide sequence of a phosphinothricin-acetyl-transferase gene is provided in EP Patent Numbers 0 242 246 and 0 242 236 to Leemans, et al. De Greef, et al., (1989) Bio/Technology 7:61 describe the production of transgenic plants that express chimeric bar genes coding for phosphinothricin acetyl transferase activity. See also, U.S. Pat. Nos. 5,969,213; 5,489,520; 5,550,318; 5,874,265; 5,919,675; 5,561,236; 5,648,477; 5,646,024; 6,177,616 B1 and 5,879,903. Exemplary genes conferring resistance to phenoxy proprionic acids and cycloshexones, such as sethoxydim and haloxyfop, are the Acc1-S1, Acc1-S2 and Acc1-S3 genes described by Marshall, et al., (1992) Theor. Appl. Genet. 83:435.

(C) A herbicide that inhibits photosynthesis, such as a triazine (psbA and gs+genes) and a benzonitrile (nitrilase gene). Przibilla, et al., (1991) Plant Cell 3:169, describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Numbers 53435, 67441 and 67442. Cloning and expression of DNA coding for a glutathione S-transferase is described by Hayes, et al., (1992) Biochem. J. 285:173.

(D) Acetohydroxy acid synthase, which has been found to make plants that express this enzyme resistant to multiple types of herbicides, has been introduced into a variety of plants (see, e.g., Hattori, et al., (1995) Mol Gen Genet 246:419). Other genes that confer resistance to herbicides include: a gene encoding a chimeric protein of rat cytochrome P4507A1 and yeast NADPH-cytochrome P450 oxidoreductase (Shiota, et al., (1994) Plant Physiol. 106:17), genes for glutathione reductase and superoxide dismutase (Aono, et al., (1995) Plant Cell Physiol 36:1687 and genes for various phosphotransferases (Datta, et al., (1992) Plant Mol Biol 20:619).

(E) Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1 and 5,767,373 and International Publication Number WO 01/12825.

### 3. Transgenes That Confer Or Contribute To an Altered Grain Characteristic, Such As:

(A) Altered fatty acids, for example, by
(1) Down-regulation of stearoyl-ACP desaturase to increase stearic acid content of the plant. See, Knultzon, et al., Proc. Natl. Acad. Sci. USA 89:2624 (1992) and WO99/64579 (Genes for Desaturases to Alter Lipid Profiles in Corn),
(2) Elevating oleic acid via FAD-2 gene modification and/or decreasing linolenic acid via FAD-3 gene modification (see, U.S. Pat. Nos. 6,063,947; 6,323,392; 6,372,965 and WO 93/11245),
(3) Altering conjugated linolenic or linoleic acid content, such as in WO 01/12800,
(4) Altering LEC1, AGP, Dek1, Superal1, mi1ps, various Ipa genes such as Ipa1, Ipa3, hpt or hggt. For example, see, WO 02/42424, WO 98/22604, WO 03/011015, U.S. Pat. Nos. 6,423,886, 6,197,561, 6,825,397, US Patent Application Publication Numbers 2003/0079247, 2003/0204870, WO02/057439, WO03/011015 and Rivera-Madrid, et al., (1995) Proc. Natl. Acad. Sci. 92:5620-5624.

(B) Altered phosphorus content, for example, by the
(1) Introduction of a phytase-encoding gene would enhance breakdown of phytate, adding more free phosphate to the transformed plant. For example, see, Van Hartingsveldt, et al., (1993) Gene 127:87, for a disclosure of the nucleotide sequence of an Aspergillus niger phytase gene.
(2) Up-regulation of a gene that reduces phytate content. In maize, this, for example, could be accomplished, by cloning and then re-introducing DNA associated with one or more of the alleles, such as the LPA alleles, identified in maize mutants characterized by low levels of phytic acid, such as in Raboy, et al., (1990) Maydica 35: 383 and/or by altering inositol kinase activity as in WO 02/059324, US Patent Application Publication Number 2003/0009011, WO 03/027243, US Patent Application Publication Number 2003/0079247, WO 99/05298, U.S. Pat. Nos. 6,197,561, 6,291,224, 6,391,348, WO2002/059324, US Patent Application Publication Number 2003/0079247, WO98/45448, WO99/55882, WO01/04147.

(C) Altered carbohydrates effected, for example, by altering a gene for an enzyme that affects the branching pattern of starch or a gene altering thioredoxin (See, U.S. Pat. No. 6,531,648). See, Shiroza, et al., (1988) J. Bacteriol. 170:810 (nucleotide sequence of Streptococcus mutans fructosyltransferase gene), Steinmetz, et al., (1985) Mol. Gen. Genet. 200:220 (nucleotide sequence of Bacillus subtilis levansucrase gene), Pen, et al., (1992) Bio/Technology 10:292 (production of transgenic plants that express Bacillus licheniformis alpha-amylase), Elliot, et al., (1993) Plant Molec. Biol. 21:515 (nucleotide sequences of tomato invertase genes), Søgaard, et al., (1993) J. Biol. Chem. 268:22480 (site-directed mutagenesis of barley alpha-amylase gene) and Fisher, et al., (1993) Plant Physiol. 102:1045 (maize endosperm starch branching enzyme II), WO 99/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned above may also be used to affect starch content and/or composition through the interrelationship of the starch and oil pathways.

(D) Altered antioxidant content or composition, such as alteration of tocopherol or tocotrienols. For example, see, U.S. Pat. No. 6,787,683, US Patent Application Publication Number 2004/0034886 and WO 00/68393 involving the manipulation of antioxidant levels through alteration of a phytl prenyl transferase (ppt), WO 03/082899 through alteration of a homogentisate geranyl transferase (hggt).

(E) Altered essential seed amino acids. For example, see, U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO99/40209 (alteration of amino acid compositions in seeds), WO99/29882 (methods for altering amino acid content of proteins), U.S. Pat. No. 5,850,016 (alteration of amino acid compositions in seeds), WO98/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO98/56935 (plant amino acid biosynthetic enzymes), WO98/45458 (engineered seed protein having higher percentage of essential amino acids), WO98/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO96/01905 (increased threonine), WO95/15392 (increased lysine), US Patent Application Publication Numbers 2003/0163838, 2003/0150014, 2004/0068767, U.S. Pat. No. 6,803,498, WO01/79516, and WO00/09706 (Ces A: cellulose synthase), U.S. Pat. No. 6,194,638 (hemicellulose), U.S. Pat. No. 6,399,859 and US Patent Application Publication Number 2004/0025203 (UDPGdH), U.S. Pat. No. 6,194,638 (RGP).

4. Genes that Control Male-sterility

There are several methods of conferring genetic male sterility available, such as multiple mutant genes at separate locations within the genome that confer male sterility, as disclosed in U.S. Pat. Nos. 4,654,465 and 4,727,219 to Brar, et al., and chromosomal translocations as described by Patterson in U.S. Pat. Nos. 3,861,709 and 3,710,511. In addition to these methods, Albertsen, et al., U.S. Pat. No. 5,432,068 describe a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant and thus creating a plant that is male sterile because the inducible promoter is not “on” resulting in the male fertility gene not being transcribed. Fertility is restored by inducing or turning “on” the promoter, which in turn allows the gene that confers male fertility to be transcribed.

(A) Introduction of a deacetylase gene under the control of a tapetum-specific promoter and with the application of the chemical N-Ac-PPT (WO 01/29237).

(B) Introduction of various stamen-specific promoters (WO 92/13956, WO 92/13957).

(C) Introduction of the barnase and the barstar gene (Paul, et al., (1992) Plant Mol. Biol. 19:611-622).

For additional examples of nuclear male and female sterility systems and genes, see also, U.S. Pat. Nos. 5,859,341; 6,297,426; 5,478,369; 5,824,524; 5,850,014 and 6,265,640.

5. Genes that create a site for site specific DNA integration.

This includes the introduction of FRT sites that may be used in the FLP/FRT system and/or Lox sites that may be used in the Cre/Loxp system. For example, see, Lyznik, et al., (2003) Plant Cell Rep 21:925-932 and WO 99/25821, which are hereby incorporated by reference. Other systems that may be used include the Gin recombinase of phage Mu (Maeser, et al., (1991) Mol Gen Genet. 230(1-2):170-6.); Vicki Chandler, The Maize Handbook ch. 118 (Springer-Verlag 1994), the Pin recombinase of E. coli (Enomoto, et al., 1983) and the R/RS system of the pSR1 plasmid (Araki, et al., (1992) J Mol. Biol. 5225(1):25-37.

6. Genes that affect abiotic stress resistance (including but not limited to flowering, ear and seed development, enhancement of nitrogen utilization efficiency, altered nitrogen responsiveness, drought resistance or tolerance, cold resistance or tolerance and salt resistance or tolerance) and increased yield under stress.

For example, see, WO 00/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, WO2000/060089, WO2001/026459, WO2001/035725, WO2001/034726, WO2001/035727, WO2001/036444, WO2001/036597, WO2001/036598, WO2002/015675, WO2002/017430, WO2002/077185, WO2002/079403, WO2003/013227, WO2003/013228, WO2003/014327, WO2004/031349, WO2004/076638, WO98/09521 and WO99/38977 describing genes, including CBF genes and transcription factors effective in mitigating the negative effects of freezing, high salinity and drought on plants, as well as conferring other positive effects on plant phenotype; US Patent Application Publication Number 2004/0148654 and WO01/36596 where abscisic acid is altered in plants resulting in improved plant phenotype such as increased yield and/or increased tolerance to abiotic stress; WO2000/006341, WO04/090143, U.S. patent application Ser. Nos. 10/817,483 and 09/545,334 where cytokinin expression is modified resulting in plants with increased stress tolerance, such as drought tolerance and/or increased yield. Also see, WO02/02776, WO2003/052063, JP2002281975, U.S. Pat. No. 6,084,153, WO0164898, U.S. Pat. No. 6,177,275 and U.S. Pat. No. 6,107,547 (enhancement of nitrogen utilization and altered nitrogen responsiveness). For ethylene alteration, see, US Patent Application Publication Numbers 2004/0128719, 2003/0166197 and WO2000/32761. For plant transcription factors or transcriptional regulators of abiotic stress, see, e.g., US Patent Application Publication Numbers 2004/0098764 or 2004/0078852.

Other genes and transcription factors that affect plant growth and agronomic traits such as yield, flowering, plant growth and/or plant structure, nutrient uptake, especially nitrogen uptake by plants, nitrogen use efficiency; drought tolerance and water use efficiency; root strength and root lodging resistance; soil pest management, corn root worm resistance can be introduced or introgressed into plants, see, e.g., WO97/49811 (LHY), WO98/56918 (ESD4), WO97/10339 and U.S. Pat. No. 6,573,430 (TFL), U.S. Pat. No. 6,713,663 (FT), WO96/14414 (CON), WO96/38560, WO01/21822 (VRN1), WO00/44918 (VRN2), WO99/49064 (GI), WO00/46358 (FRI), WO97/29123, U.S. Pat. Nos. 6,794,560, 6,307,126 (GAI), WO99/09174 (D8 and Rht) and WO2004/076638 and WO2004/031349 (transcription factors).

Commercial traits in plants can be created through the expression of genes that alter starch or protein for the production of paper, textiles, ethanol, polymers or other materials with industrial uses.

Means of increasing or inhibiting a protein are well known to one skilled in the art and, by way of example, may include, transgenic expression, antisense suppression, co-suppression methods including but not limited to: RNA interference, gene activation or suppression using transcription factors and/or repressors, mutagenesis including transposon tagging, directed and site-specific mutagenesis, chromosome engineering (see, Nobrega, et. al., (2004) Nature 431:988-993), homologous recombination, TILLING (Targeting Induced Local Lesions In Genomes) and biosynthetic competition to manipulate, the expression of proteins.

Many techniques for gene silencing are well known to one of skill in the art, including but not limited to knock-outs (such as by insertion of a transposable element such as Mu, Chandler, The Maize Handbook ch. 118 (Springer-Verlag 1994) or other genetic elements such as a FRT, Lox or other site specific integration site; RNA interference (Napoli, et al., (1990) Plant Cell 2:279-289; U.S. Pat. No. 5,034,323, Sharp (1999) Genes Dev. 13:139-141, Zamore, et al., (2000) Cell 101:25-33 and Montgomery, et al., (1998) PNAS USA 95:15502-15507); virus-induced gene silencing (Burton, et al., (2000) Plant Cell 12:691-705 and Baulcombe, (1999) Curr. Op. Plant Bio. 2:109-113); target-RNA-specific ribozymes (Haseloff, et al., (1988) Nature 334:585-591); hairpin structures (Smith, et al., (2000) Nature 407:319-320; WO 99/53050 and WO 98/53083); MicroRNA (Aukerman and Sakai, (2003) Plant Cell 15:2730-2741); ribozymes (Steinecke, et al., (1992) EMBO J. 11:1525 and Perriman, et al., (1993) Antisense Res. Dev. 3:253); oligonucleotide mediated targeted modification (e.g., WO 03/076574 and WO 99/25853); zinc-finger targeted molecules (e.g., WO 01/52620; WO 03/048345 and WO 00/42219) and other methods or combinations of the above methods known to those of skill in the art.

Any method of increasing or inhibiting a protein can be used in the present invention. Several examples are outlined in more detail below for illustrative purposes.

The nucleotide sequence operably linked to the regulatory elements disclosed herein can be an antisense sequence for a targeted gene. (See, e.g., Sheehy, et al., (1988) PNAS USA 85:8805-8809 and U.S. Pat. Nos. 5,107,065; 5,453,566 and 5,759,829). By “antisense DNA nucleotide sequence” is intended a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing with the endogenous messenger RNA (mRNA) produced by transcription of the DNA nucleotide sequence for the targeted gene. In this case, production of the native protein encoded by the targeted gene is inhibited to achieve a desired phenotypic response. Thus the regulatory sequences disclosed herein can be operably linked to antisense DNA sequences to reduce or inhibit expression of a native protein in the plant root.

As noted, other potential approaches to impact expression of proteins in the root include traditional co-supression, that is, inhibition of expression of an endogenous gene through the expression of an identical structural gene or gene fragment introduced through transformation (Goring, et al., (1991) Proc. Natl. Acad. Sci. USA 88:1770-1774 co-suppression; Taylor, (1997) Plant Cell 9:1245; Jorgensen, (1990) Trends Biotech. 8(12):340-344; Flavell, (1994) PNAS USA 91:3490-3496; Finnegan, et al., (1994) Bio/Technology 12:883-888 and Neuhuber, et al., (1994) Mol. Gen. Genet. 244:230-241). In one example, co-suppression can be achieved by linking the promoter to a DNA segment such that transcripts of the segment are produced in the sense orientation and where the transcripts have at least 65% sequence identity to transcripts of the endogenous gene of interest, thereby suppressing expression of the endogenous gene in said plant cell. (See, U.S. Pat. No. 5,283,184). The endogenous gene targeted for co-suppression may be a gene encoding any protein that accumulates in the plant species of interest. For example, where the endogenous gene targeted for co-suppression is the 50 kD gamma-zein gene, co-suppression is achieved using an expression cassette comprising the 50 kD gamma-zein gene sequence or variant or fragment thereof.

Additional methods of co-suppression are known in the art and can be similarly applied to the instant invention. These methods involve the silencing of a targeted gene by spliced hairpin RNA's and similar methods also called RNA interference and promoter silencing (see, Smith, et al., (2000) Nature 407:319-320, Waterhouse and Helliwell, (2003) Nat. Rev. Genet. 4:29-38; Waterhouse, et al., (1998) Proc. Natl. Acad. Sci. USA 95:13959-13964; Chuang and Meyerowitz, (2000) Proc. Natl. Acad. Sci. USA 97:4985-4990; Stoutjesdijk, et al., (2002) Plant Physiol. 129:1723-1731 and Patent Application WO 99/53050; WO 99/49029; WO 99/61631; WO 00/49035 and U.S. Pat. No. 6,506,559.

For mRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). miRNA molecules are highly efficient at inhibiting the expression of endogenous genes and the RNA interference they induce is inherited by subsequent generations of plants.

In one embodiment, the polynucleotide to be introduced into the plant comprises an inhibitory sequence that encodes a zinc finger protein that binds to a gene encoding a protein of the invention resulting in reduced expression of the gene. In particular embodiments, the zinc finger protein binds to a regulatory region of a gene of the invention. In other embodiments, the zinc finger protein binds to a messenger RNA encoding a protein and prevents its translation. Methods of selecting sites for targeting by zinc finger proteins have been described, for example, in U.S. Pat. No. 6,453,242 and methods for using zinc finger proteins to inhibit the expression of genes in plants are described, for example, in US Patent Application Publication Number 2003/0037355.

The expression cassette may also include at the 3′ terminus of the isolated nucleotide sequence of interest, a transcriptional and translational termination region functional in plants. The termination region can be native with the promoter nucleotide sequence of the present invention, can be native with the DNA sequence of interest or can be derived from another source.

Any convenient termination regions can be used in conjunction with the promoter of the invention and are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; Joshi, et al., (1987) Nucleic Acid Res. 15:9627-9639.

The expression cassettes can additionally contain 5′ leader sequences. Such leader sequences can act to enhance translation. Translation leaders are known in the art and include: picornavirus leaders, for example, EMCV leader (Encephalomyocarditis 5′ noncoding region), Elroy-Stein, et al., (1989) Proc. Natl. Acad. Sci. USA 86:6126-6130; potyvirus leaders, for example, TEV leader (Tobacco Etch Virus), Allison, et al., (1986); Virology 154:9-20; human immunoglobulin heavy-chain binding protein (BiP), Macejak, et al., (1991) Nature 353:90-94; untranslated leader from the coat protein mRNA of alfalfa mosaic virus (AMV RNA 4), Jobling, et al., (1987) Nature 325:622-625); tobacco mosaic virus leader (TMV), Gallie, et al., (1989) Molecular Biology of RNA, pages 237-256 and maize chlorotic mottle virus leader (MCMV), Lommel, et al., (1991) Virology 81:382-385. See also, Della-Cioppa, et al., (1987) Plant Physiology 84:965-968. The cassette can also contain sequences that enhance translation and/or mRNA stability such as introns.

In those instances where it is desirable to have an expressed product of an isolated nucleotide sequence directed to a particular organelle, particularly the plastid, amyloplast or to the endoplasmic reticulum or secreted at the cell's surface or extracellularly, the expression cassette can further comprise a coding sequence for a transit peptide. Such transit peptides are well known in the art and include, but are not limited to: the transit peptide for the acyl carrier protein, the small subunit of RUBISCO, plant EPSP synthase and the like.

In preparing the expression cassette, the various DNA fragments can be manipulated, so as to provide for the DNA sequences in the proper orientation and, as appropriate, in the proper reading frame. Toward this end, adapters or linkers can be employed to join the DNA fragments or other manipulations can be involved to provide for convenient restriction sites, removal of superfluous DNA, removal of restriction sites or the like. For this purpose, in vitro mutagenesis, primer repair, restriction digests, annealing and resubstitutions such as transitions and transversions, can be involved.

As noted herein, the present invention provides vectors capable of expressing genes of interest under the control of the regulatory elements. In general, the vectors should be functional in plant cells. At times, it may be preferable to have vectors that are functional in E. coli (e.g., production of protein for raising antibodies, DNA sequence analysis, construction of inserts, obtaining quantities of nucleic acids). Vectors and procedures for cloning and expression in E. coli are discussed in Sambrook, et al., (supra).

The transformation vector comprising the regulatory sequences of the present invention operably linked to an isolated nucleotide sequence in an expression cassette, can also contain at least one additional nucleotide sequence for a gene to be cotransformed into the organism. Alternatively, the additional sequence(s) can be provided on another transformation vector.

Vectors that are functional in plants can be binary plasmids derived from Agrobacterium. Such vectors are capable of transforming plant cells. These vectors contain left and right border sequences that are required for integration into the host (plant) chromosome. At minimum, between these border sequences is the gene to be expressed under control of the regulatory elements of the present invention. In one embodiment, a selectable marker and a reporter gene are also included. For ease of obtaining sufficient quantities of vector, a bacterial origin that allows replication in E. coli can be used.

Reporter genes can be included in the transformation vectors. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) BioTechniques 19:650-655 and Chiu, et al., (1996) Current Biology 6:325-330.

Selectable marker genes for selection of transformed cells or tissues can be included in the transformation vectors. These can include genes that confer antibiotic resistance or resistance to herbicides. Examples of suitable selectable marker genes include, but are not limited to: genes encoding resistance to chloramphenicol, Herrera Estrella, et al., (1983) EMBO J. 2:987-992; methotrexate, Herrera Estrella, et al., (1983) Nature 303:209-213; Meijer, et al., (1991) Plant Mol. Biol. 16:807-820; hygromycin, Waldron, et al., (1985) Plant Mol. Biol. 5:103-108; Zhijian, et al., (1995) Plant Science 108:219-227; streptomycin, Jones, et al., (1987) Mol. Gen. Genet. 210:86-91; spectinomycin, Bretagne-Sagnard, et al., (1996) Transgenic Res. 5:131-137; bleomycin, Hille, et al., (1990) Plant Mol. Biol. 7:171-176; sulfonamide, Guerineau, et al., (1990) Plant Mol. Biol. 15:127-136; bromoxynil, Stalker, et al., (1988) Science 242:419-423; glyphosate, Shaw, et al., (1986) Science 233:478-481; phosphinothricin, DeBlock, et al., (1987) EMBO J. 6:2513-2518.

Further, when linking a promoter of the invention with a nucleotide sequence encoding a detectable protein, expression of a linked sequence can be tracked in the plant, thereby providing a useful so-called screenable or scorable markers. The expression of the linked protein can be detected without the necessity of destroying tissue. More recently, interest has increased in utilization of screenable or scorable markers. By way of example without limitation, the promoter can be linked with detectable markers including a β-glucuronidase or uidA gene (GUS) which encodes an enzyme for which various chromogenic substrates are known (Jefferson, et al., (1986) Proc. Natl. Acad. Sci. USA 83:8447-8451); chloramphenicol acetyl transferase; alkaline phosphatase; a R-locus gene, which encodes a product that regulates the production of anthocyanin pigments (red color) in plant tissues (Dellaporta, et al., in Chromosome Structure and Function, Kluwer Academic Publishers, Appels and Gustafson, eds., pp. 263-282 (1988); Ludwig, et al., (1990) Science 247:449); a p-lactamase gene (Sutcliffe, (1978) Proc. Nat'l. Acad. Sci. U.S.A. 75:3737), which encodes an enzyme for which various chromogenic substrates are known (e.g., PADAC, a chromogenic cephalosporin); a xylE gene (Zukowsky, et al., (1983) Proc. Nat'l. Acad. Sci. U.S.A. 80:1101), which encodes a catechol dioxygenase that can convert chromogenic catechols; an α-amylase gene (Ikuta, et al., (1990) Biotech. 8:241); a tyrosinase gene (Katz, et al., (1983) J. Gen. Microbiol. 129:2703), which encodes an enzyme capable of oxidizing tyrosine to DOPA and dopaquinone, which in turn condenses to form the easily detectable compound melanin a green fluorescent protein (GFP) gene (Sheen, et al., (1995) Plant J. 8(5):777-84); a lux gene, which encodes a luciferase, the presence of which may be detected using, for example, X-ray film, scintillation counting, fluorescent spectrophotometry, low-light video cameras, photon counting cameras or multiwell luminometry (Teeri, et al., (1989) EMBO J. 8:343); DS-RED EXPRESS (Matz, et al., (1999) Nature Biotech. 17:969-973, Bevis, et al., (2002) Nature Biotech 20:83-87, Haas, et al., (1996) Curr. Biol. 6:315-324); Zoanthus sp. yellow fluorescent protein (ZsYellow) that has been engineered for brighter fluorescence (Matz, et al., (1999) Nature Biotech. 17:969-973, available from BD Biosciences Clontech, Palo Alto, Calif., USA, Catalog Number K6100-1) and cyan florescent protein (CYP) (Bolte, et al., (2004) J. Cell Science 117:943-54 and Kato, et al., (2002) Plant Physiol 129:913-42).

A transformation vector comprising the particular regulatory sequences of the present invention, operably linked to an isolated nucleotide sequence of interest in an expression cassette, can be used to transform any plant. In this manner, genetically modified plants, plant cells, plant tissue, root and the like can be obtained. Transformation protocols can vary depending on the type of plant or plant cell, i.e., monocot or dicot, targeted for transformation. Suitable methods of transforming plant cells include microinjection, Crossway, et al., (1986) Biotechniques 4:320-334; electroporation, Riggs, et al., (1986) Proc. Natl. Acad. Sci. USA 83:5602-5606; Agrobacterium-mediated transformation, see for example, Townsend, et al., U.S. Pat. No. 5,563,055; direct gene transfer, Paszkowski, et al., (1984) EMBO J. 3:2717-2722 and ballistic particle acceleration, see for example, Sanford, et al., U.S. Pat. No. 4,945,050, Tomes, et al., (1995) in Plant Cell, Tissue, and Organ Culture: Fundamental Methods, ed. Gamborg and Phillips, (Springer-Verlag, Berlin) and McCabe, et al., (1988) Biotechnology 6:923-926. Also, see, Weissinger, et al., (1988) Annual Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Datta, et al., (1990) Bio/Technology 8:736-740 (rice); Klein, et al., (1988) Proc. Natl. Acad. Sci. USA 85:4305-4309 (maize); Klein, et al., (1988) Biotechnology 6:559-563 (maize); Klein, et al., (1988) Plant Physiol. 91:440-444 (maize); Fromm, et al., (1990) Biotechnology 8:833-839; Hooydaas-Van Slogteren, et al., (1984) Nature (London) 311:763-764; Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, N.Y.), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D. Halluin, et al., (1992) Plant Cell 4:1495-1505 (electroporation); Li, et al., (1993) Plant Cell Reports 12:250-255 and Christou, et al., (1995) Annals of Botany 75:407-413 (rice); Osjoda, et al., (1996) Nature Biotechnology 14:745-750 (maize via Agrobacterium tumefaciens).

The cells that have been transformed can be grown into plants in accordance with conventional methods. See, for example, McCormick, et al., (1986) Plant Cell Reports 5:81-84. These plants can then be grown and pollinated with the same transformed strain or different strains. The resulting plant having root-preferred expression of the desired phenotypic characteristic can then be identified. Two or more generations can be grown to ensure that root-preferred expression of the desired phenotypic characteristic is stably maintained and inherited.

The following examples are offered by way of illustration and not by way of limitation.

### EXAMPLES

### Example 1

### Prediction of Expression via Lynx MPSS

Lynx™ gene expression profiling technology was used to identify the maize Zm-SARK coding region as a candidate for promoter isolation. Massively parallel signature sequencing (MPSS, see, Brenner. et al., (2000) Nature Biotechnology 18:630-634) indicated expression in various genotypes in root and other tissues. Expression was observed in a variety of maize tissues. MPSS data showed highest expression of maize SARK in root and leaf tissue.

### Example 2

### Prediction of Expression Pattern via RT PCR

RT-PCR can be performed on maize tissues from seedlings and mature plants, separated to roots and shoots, as well as more mature tissue. Results as shown by gel electrophoresis would compare with the MPSS data. The RT-PCR data indicates the expression pattern throughout the plant over time.

### Example 3

### Transformation of Maize by Particle Bombardment

### Preparation of Particles

Sixty mg of 0.6 u BioRad gold particles was weighed and placed in a 2 ml microfuge tube. 1 ml of 100% EtOH was added to the gold particles and sonicated briefly (Branson Sonifier Model 450, 40% output, constant duty cycle), the vortexed on high for 1 minute. The gold particles were pelleted by centrifugation at 10000 rpm (Biofuge) for one minute and the EtOH was withdrawn. This EtOH wash was repeated two more times. After the last centrifugation, the 100% EtOH was withdrawn and replaced with 1 ml sterile deionized water and briefly sonicated. The solution was then aliquotted into 250 ul aliquots and 750 ul of sterile deionized water was added to each aliquot.

### Preparation of Particle-Plasmid DNA Association

100 ul of the tungsten particle (0.6 u gold particles) solution was briefly sonicated. 10 ul of plasmid DNA (100 ng/ul), 100 μl 2.5 M CaCl₂ and 10 μl 0.1 M spermidine was added and vortexed for 10 minutes at a medium speed.

After the association period, the tubes were centrifuged briefly, liquid removed, washed with 500 μl 100% ethanol by sonicating for 3 seconds and centrifuging for 30 seconds. Again the liquid was removed and 105 μl of 100% ethanol added to the final tungsten pellet. The associated particles/DNA were briefly sonicated and 10 μl spotted onto the center of each macro-carrier and allowed to dry ˜2 minutes before bombardment.

### Preparation of Target Seedling Roots

B73 seeds were placed along one edge of growth paper soaked in water. An additional piece of growth paper identical in size to the first was also soaked in water and overlaid onto the seeds. The growth paper-seed-growth paper sandwich was subsequently jelly rolled with the seed edge at the top of the roll. The roll was directionally placed into a beaker of water with the seeds at the top to allow for straight root growth. Seeds were allowed to germinate and develop for 2-3 days in the dark at 28° C. Prior to bombardment the outer skin layer of the cotyledon was removed and seedlings were placed in a sterile petri dish (60 mm) on a layer of Whatman® #1 filter paper moistened with 1 mL of water. Two seedlings per plate were arranged in opposite orientations and anchored to the filter paper with a 0.5% agarose solution. 2-3 cm root tip sections were also excised from seedlings and arranged lengthwise in the plates for bombardment.

### Particle Bombardment

To effect particle bombardment of roots, the particle-DNA agglomerates were accelerated using a DuPont PDS-1000 particle acceleration device. The particle-DNA agglomeration was briefly sonicated and 10 μl were deposited on macrocarriers and the ethanol allowed to evaporate. The macrocarrier was accelerated onto a stainless-steel stopping screen by the rupture of a polymer diaphragm (rupture disk). Rupture is effected by pressurized helium. The velocity of particle-DNA acceleration is determined based on the rupture disk breaking pressure. A rupture disk pressure of 1100 psi was used.

The shelf containing the plate with the roots was placed 5.1 cm below the bottom of the macrocarrier platform (shelf #3). To effect particle bombardment of the roots, a rupture disk and a macrocarrier with dried particle-DNA agglomerates were installed in the device. The He pressure delivered to the device was adjusted to 200 psi above the rupture disk breaking pressure. A Petri dish with the target kernels was placed into the vacuum chamber and located in the projected path of accelerated particles. A vacuum was created in the chamber, preferably about 28 in Hg. After operation of the device, the vacuum was released and the Petri dish removed. Bombarded roots would be analyzed for expression 18-24 hours after bombardment. Ability of the promoter to drive expression in maize root from 2-3 days after germination would be confirmed by GUS detection in of root of bombarded kernels.

### Example 4

### Transformation and Regeneration of Maize Callus via Agrobacterium

Constructs used were as those set forth supra for microprojectile bombardment, except that the control was not employed in this experiment and the selectable marker for maize-optimized PAT (phosphinothricin acetyl transferase) was also included.

### Preparation of Agrobacterium Suspension

Agrobacterium was streaked out from a −80° C. frozen aliquot onto a plate containing PHI-L medium and was cultured at 28° C. in the dark for 3 days. PHI-L media comprises 25 ml/I Stock Solution A, 25 ml/I Stock Solution B, 450.9 ml/I Stock Solution C and spectinomycin (Sigma Chemicals) was added to a concentration of 50 mg/l in sterile ddH2O (stock solution A: K₂HPO₄ 60.0 g/l, NaH₂PO₄ 20.0 g/l, adjust pH to 7.0 w/KOH and autoclaved; stock solution B: NH₄Cl 20.0 g/l, MgSO₄.7H₂O 6.0 g/l, KCl 3.0 g/l, CaCl₂ 0.20 g/l, FeSO₄.7H₂O 50.0 mg/l, autoclaved; stock solution C: glucose 5.56 g/l, agar 16.67 g/l (#A-7049, Sigma Chemicals, St. Louis, Mo.) and was autoclaved).

The plate can be stored at 4° C. and used usually for about 1 month. A single colony was picked from the master plate and was streaked onto a plate containing PHI-M medium [yeast extract (Difco) 5.0 g/l; peptone (Difco) 10.0 g/l; NaCl 5.0 g/l; agar (Difco) 15.0 g/l; pH 6.8, containing 50 mg/L spectinomycin] and was incubated at 28° C. in the dark for 2 days.

Five ml of either PHI-A, [CHU(N6) basal salts (Sigma C-1416) 4.0 g/l, Eriksson's vitamin mix (1000×, Sigma-1511) 1.0 ml/l; thiamine.HCl 0.5 mg/l (Sigma); 2,4-dichlorophenoxyacetic acid (2,4-D, Sigma) 1.5 mg/l; L-proline (Sigma) 0.69 g/l; sucrose (Mallinckrodt) 68.5 g/l; glucose (Mallinckrodt) 36.0 g/l; pH 5.2] for the PHI basic medium system, or PHI-I [MS salts (GIBCO BRL) 4.3 g/l; nicotinic acid (Sigma) 0.5 mg/l; pyridoxine.HCl (Sigma) 0.5 mg/l; thiamine.HCl 1.0 mg/l; myo-inositol (Sigma) 0.10 g/l; vitamin assay casamino acids (Difco Lab) 1 g/l; 2,4-D1.5 mg/l; sucrose 68.50 g/l; glucose 36.0 g/l; adjust pH to 5.2 w/KOH and filter-sterilize] for the PHI combined medium system and 5 ml of 100 mM (3′-5′-Dimethoxy-4′-hydroxyacetophenone, Aldrich chemicals) was added to a 14 ml Falcon tube in a hood. About 3 full loops (5 mm loop size) Agrobacterium was collected from the plate and suspended in the tube, then the tube vortexed to make an even suspension. One ml of the suspension was transferred to a spectrophotometer tube and the OD of the suspension is adjusted to 0.72 at 550 nm by adding either more Agrobacterium or more of the same suspension medium, for an Agrobacterium concentration of approximately 0.5×109 cfu/ml to 1×109 cfu/ml. The final Agrobacterium suspension was aliquoted into 2 ml microcentrifuge tubes, each containing 1 ml of the suspension. The suspensions were then used as soon as possible.

### Embryo Isolation, Infection and Co-Cultivation

About 2 ml of the same medium (here PHI-A or PHI-I) which is used for the Agrobacterium suspension was added into a 2 ml microcentrifuge tube. Immature embryos were isolated from a sterilized ear with a sterile spatula (Baxter Scientific Products S1565) and dropped directly into the medium in the tube. A total of about 100 embryos are placed in the tube. The optimal size of the embryos was about 1.0-1.2 mm. The cap was then closed on the tube and the tube vortexed with a Vortex Mixer (Baxter Scientific Products S8223-1) for 5 sec. at maximum speed. The medium was removed and 2 ml of fresh medium were added and the vortexing repeated. All of the medium was drawn off and 1 ml of Agrobacterium suspension was added to the embryos and the tube is vortexed for 30 sec. The tube was allowed to stand for 5 min. in the hood. The suspension of Agrobacterium and embryos was poured into a Petri plate containing either PHI-B medium [CHU(N6) basal salts (Sigma C-1416) 4.0 g/l; Eriksson's vitamin mix (1000×, Sigma-1511) 1.0 ml/l; thiamine.HCl 0.5 mg/l; 2.4-D1.5 mg/l; L-proline 0.69 g/l; silver nitrate 0.85 mg/l; Gelrite® (Sigma) 3.0 g/l; sucrose 30.0 g/l; acetosyringone 100 mM; pH 5.8], for the PHI basic medium system, or PHI-J medium [MS Salts 4.3 g/l; nicotinic acid 0.50 mg/l; pyridoxine HCl 0.50 mg/l; thiamine.HCl 1.0 mg/l; myo-inositol 100.0 mg/l; 2,4-D1.5 mg/l; sucrose 20.0 g/l; glucose 10.0 g/l; L-proline 0.70 g/l; MES (Sigma) 0.50 g/l; 8.0 g/l agar (Sigma A-7049, purified) and 100 mM acetosyringone with a final pH of 5.8 for the PHI combined medium system. Any embryos left in the tube were transferred to the plate using a sterile spatula. The Agrobacterium suspension was drawn off and the embryos placed axis side down on the media. The plate was sealed with Parafilm® tape or Pylon Vegetative Combine Tape (product named “E.G.CUT” and is available in 18 mm×50 m sections; Kyowa Ltd., Japan) and was incubated in the dark at 23-25° C. for about 3 days of co-cultivation.

### Resting, Selection and Regeneration Steps

For the resting step, all of the embryos were transferred to a new plate containing PHI-C medium [CHU(N6) basal salts (Sigma C-1416) 4.0 g/l; Eriksson's vitamin mix (1000× Sigma-1511) 1.0 ml/l; thiamine.HCl 0.5 mg/l; 2.4-D1.5 mg/l; L-proline 0.69 g/l; sucrose 30.0 g/l; MES buffer (Sigma) 0.5 g/l; agar (Sigma A-7049, purified) 8.0 g/l; silver nitrate 0.85 mg/l; carbenicillin 100 mg/l; pH 5.8]. The plate was sealed with Parafilm® or Pylon tape and incubated in the dark at 28° C. for 3-5 days.

Longer co-cultivation periods may compensate for the absence of a resting step since the resting step, like the co-cultivation step, provides a period of time for the embryo to be cultured in the absence of a selective agent. Those of ordinary skill in the art can readily test combinations of co-cultivation and resting times to optimize or improve the transformation.

For selection, all of the embryos were then transferred from the PHI-C medium to new plates containing PHI-D medium, as a selection medium, [CHU(N6) basal salts (SIGMA C-1416) 4.0 g/l; Eriksson's vitamin mix (1000×, Sigma-1511) 1.0 ml/l; thiamine.HCl 0.5 mg/l; 2.4-D1.5 mg/l; L-proline 0.69 g/l; sucrose 30.0 g/l; MES buffer 0.5 g/l; agar (Sigma A-7049, purified) 8.0 g/l; silver nitrate 0.85 mg/l; carbenicillin (ICN, Costa Mesa, Calif.) 100 mg/l; bialaphos (Meiji Seika K. K., Tokyo, Japan) 1.5 mg/l for the first two weeks followed by 3 mg/l for the remainder of the time; pH 5.8] putting about 20 embryos onto each plate.

The plates were sealed as described above and incubated in the dark at 28° C. for the first two weeks of selection. The embryos were transferred to fresh selection medium at two-week intervals. The tissue was subcultured by transferring to fresh selection medium for a total of about 2 months. The herbicide-resistant calli are then “bulked up” by growing on the same medium for another two weeks until the diameter of the calli is about 1.5-2 cm.

For regeneration, the calli were then cultured on PHI-E medium [MS salts 4.3 g/l; myo-inositol 0.1 g/l; nicotinic acid 0.5 mg/l, thiamine.HCl 0.1 mg/l, Pyridoxine.HCl 0.5 mg/l, Glycine 2.0 mg/l, Zeatin 0.5 mg/l, sucrose 60.0 g/l, Agar (Sigma, A-7049) 8.0 g/l, Indoleacetic acid (IAA, Sigma) 1.0 mg/l, Abscisic acid (ABA, Sigma) 0.1 mM, Bialaphos 3 mg/l, carbenicillin 100 mg/l adjusted to pH 5.6] in the dark at 28° C. for 1-3 weeks to allow somatic embryos to mature. The calli were then cultured on PHI-F medium (MS salts 4.3 g/l; myo-inositol 0.1 g/l; Thiamine.HCl 0.1 mg/l, Pyridoxine.HCl 0.5 mg/l, Glycine 2.0 mg/l, nicotinic acid 0.5 mg/l; sucrose 40.0 g/l; Gelrite® 1.5 g/l; pH 5.6] at 25° C. under a daylight schedule of 16 hrs. light (270 uE m-2sec-1) and 8 hrs. dark until shoots and roots are developed. Each small plantlet was then transferred to a 25×150 mm tube containing PHI-F medium and is grown under the same conditions for approximately another week. The plants were transplanted to pots with soil mixture in a greenhouse. DS-RED EXPRESS events are determined at the callus stage or regenerated plant stage.

Ability of the promoter to drive expression in maize root from seedlings would be confirmed by GUS detection in plant root tissue by the procedures outlined supra.

### Example 5

### Diurnal Expression Pattern of the Maize ZM-SARK Gene

The day-night cycle is a major environmental cue that controls daily and seasonal rhythms in plants. Diurnal light-dark transitions entrain the internal circadian clock that generates rhythms that are self-sustained (free-running) under constant light conditions. The proper synchronization of the internal clock and external light/dark cycles result in better plant fitness, survival, competitive advantage and growth vigor.

The day-night cycle is a major contributor to gene expression patterns, with greater than 20% of all transcripts undergoing significant cycling. Proprietary in-house RNA profiling microarray data was used for detecting diurnal expression of the ZM-SARK gene. RNA profiling was performed on custom Agilent Maize arrays designed to interrogate global gene expression patterns across 105K probes. B73 maize plants were grown under field conditions and sampled the reproductive growth at the V14-15 stage. Light conditions at the time of sampling were approximately 14.75 hours of sunlight according to records of US Naval Observatory (Materials and Methods). Starting at sunrise on day 1, top leaf and immature ear were sampled at 4 hour time intervals over three consecutive days. The ZM-SARK gene was represented by the 60-mer probe GAGAACAACCTACTCGTACCTTGAGTCCATTGTATGTAGTAATTAATGTGTTTACTAC AT (SEQ ID NO: 2) on the arrays. This probe showed diurnal pattern of expression in leaves with peak of expression during the night hours between 10 pm and 2 am. During the day the low expression was found at 2 pm. In the maize reproductive tissue, the ear, ZM-SARK is expressed but lacking the diurnal pattern. This result indicated ZM-SARK is diurnal regulated in photosynthetic tissues (leaves) and disconnected from the circadian clock in non-photosynthetic tissues (the ears).

Although the foregoing invention has been described in some detail by way of illustration and example for purposes of clarity of understanding, it will be obvious that certain changes and modifications may be practiced within the scope of the appended claims. All references cited are incorporated herein by reference.

That which is claimed:

1. An isolated nucleic acid molecule comprising a polynucleotide which initiates transcription in a plant cell and comprises a sequence selected from the group consisting of:
a) SEQ ID NO: 1;
b) at least 70 contiguous nucleotides of SEQ ID NO: 1;
c) a sequence having at least 70% sequence identity to the full length of SEQ ID NO: 1;
d) a sequence of a polynucleotide that hybridizes under stringent conditions to the complement of SEQ ID NO: 1.

2. An expression cassette comprising a polynucleotide of claim 1 operably linked to a polynucleotide of interest.

3. A vector comprising the expression cassette of claim 2.

4. A plant cell having stably incorporated into its genome the expression cassette of claim 2.

5. The plant cell of claim 4, wherein said plant cell is from a monocot.

6. The plant cell of claim 5, wherein said monocot is maize, barley, wheat, oat, rye, sorghum or rice.

7. A plant having stably incorporated into its genome the expression cassette of claim 2.

8. The plant of claim 7, wherein said plant is a monocot.

9. The plant of claim 8, wherein said monocot is maize, barley, wheat, oat, rye, sorghum or rice.

10. A transgenic seed of the plant of claim 7.

11. The plant of claim 7, wherein the polynucleotide of interest encodes a gene product that confers pathogen or insect resistance.

12. The plant of claim 7, wherein the polynucleotide of interest encodes a polypeptide involved in nutrient uptake, nitrogen use efficiency, drought tolerance, root strength, root lodging resistance, soil pest management, corn root worm resistance, carbohydrate metabolism, protein metabolism, fatty acid metabolism or phytohormone biosynthesis.

13. A method for expressing a first polynucleotide in a plant, said method comprising introducing into a plant an expression cassette comprising a promoter and a first polynucleotide operably linked thereto, wherein said promoter comprises a second polynucleotide that initiates transcription of an operably linked polynucleotide in a plant cell, and wherein said second polynucleotide comprises a sequence selected from the group consisting of:
a) SEQ ID NO: 1;
b) at least 70 contiguous nucleotides of SEQ ID NO: 1;
c) a sequence with at least 70% sequence identity to SEQ ID NO: 1; and
d) a sequence of a polynucleotide that hybridizes under stringent conditions to the complement of SEQ ID NO: 1.

14. The method of claim 13, wherein said first polynucleotide is expressed in the transformed plant.

15. The method of claim 13, wherein said plant is a monocot.

16. The method of claim 15, wherein said monocot is maize, barley, wheat, oat, rye, sorghum or rice.

17. The method of claim 13, wherein said first polynucleotide encodes a gene product that confers pathogen or insect resistance.

18. The method of claim 13, wherein said first polynucleotide encodes a polypeptide involved in nutrient uptake, nitrogen use efficiency, drought tolerance, root strength, root lodging resistance, soil pest management, corn root worm resistance, carbohydrate metabolism, protein metabolism, fatty acid metabolism or phytohormone biosynthesis.

19. A method for expressing a first polynucleotide in a plant cell, said method comprising introducing into a plant cell an expression cassette comprising a promoter and a first polynucleotide operably linked thereto, wherein said promoter comprises a second polynucleotide that initiates transcription of an operably linked polynucleotide in a plant cell, and wherein said second polynucleotide is selected from the group consisting of:
a) a polynucleotide comprising the sequence set forth in SEQ ID NO: 1, or a complement thereof;
b) a polynucleotide comprising at least 70 contiguous nucleotides of the sequence set forth in SEQ ID NO: 1;
c) a polynucleotide comprising a sequence having at least 70% sequence identity to the sequence set forth in SEQ ID NO: 1; and
d) a polynucleotide that hybridizes under stringent conditions to the complement of SEQ ID NO: 1.

20. The method of claim 19, wherein said plant cell is from a monocot.

21. The method of claim 20, wherein said monocot is maize, barley, wheat, oat, rye, sorghum or rice.

22. The method of claim 19, wherein said first polynucleotide encodes a gene product that confers pathogen or insect resistance.

23. The method of claim 19, wherein said first polynucleotide encodes a polypeptide involved in nutrient uptake, nitrogen use efficiency, drought tolerance, root strength, root lodging resistance, soil pest management, corn root worm resistance, carbohydrate metabolism, protein metabolism, fatty acid metabolism or phytohormone biosynthesis.

24. A method for selectively expressing a first polynucleotide in the root of a plant, said method comprising introducing into a plant an expression cassette comprising a promoter and a first polynucleotide operably linked thereto, wherein said promoter comprises a second polynucleotide that initiates transcription of an operably linked polynucleotide in the root of a plant and wherein said second polynucleotide is selected from the group consisting of:
a) a polynucleotide comprising the sequence set forth in SEQ ID NO: 1, or a complement thereof;
b) a polynucleotide comprising at least 70 contiguous nucleotides of the sequence set forth in SEQ ID NO: 1;
c) a polynucleotide comprising a sequence having at least 70% sequence identity to the sequence set forth in SEQ ID NO: 1; and
d) a polynucleotide sequence that hybridizes under stringent conditions to the complement of SEQ ID NO: 1.

25. The method of claim 24, wherein expression of said first polynucleotide alters the phenotype of said transformed seed.

26. The method of claim 24, wherein the plant is a monocot.

27. The method of claim 26, wherein the monocot is maize, barley, wheat, oat, rye, sorghum or rice.

28. The method of claim 24, wherein the first polynucleotide encodes a gene product that confers pathogen or insect resistance.

29. The method of claim 24, wherein the first polynucleotide encodes a polypeptide involved in nutrient uptake, nitrogen use efficiency, drought tolerance, root strength, root lodging resistance, soil pest management, corn root worm resistance, carbohydrate metabolism, protein metabolism, fatty acid metabolism or phytohormone biosynthesis.

30. A method of altering plant phenotype comprising:
(a) transforming a plant host cell with at least one isolated nucleic acid molecule of claim 1 operably linked to at least one polynucleotide of interest;
(b) growing the transformed host cell under conditions favoring plant regeneration; and
(c) generating a plant wherein said regenerated plant exhibits an altered phenotype.


SEQUENCE LISTING (7 sequences)

SEQ ID NO: 1 - DNA, 2129 bp, Zea mays
ggaaggcacacagcgttacaaggcacggacgctcaagttcgcacacaagctctcaaccacacagacacaggtttgacacatactcaatgctacatacacacagtggaggtagggtattacgctccgacggcctgaaccactctaatccctcgtgtcctcgtgtgttcatcccaaatccacataacaggtaaaacgcttaggccccctcctcatcttaggattagggcgggtgcattccgtcacccggccggtgaatttctcccaccgacagatactttggcttgttaataaactagtgatataagaggctaattggcgagacattggtttataaggactccattatatagctatctagatattaacttaaattttcctttgtagttatatacgatgtattgtaacttgtgaaaaatgtttggtgcgtgtgagaaatgacaatgtggtattggacctatagtgacaattgacaatccactatcgtgtgatccggcactagtgtcatgagtataattgttgggttgttgtctaaactggtagtatcactcaggataccactaacggtttaagatgatatctggtaatgatatgtataaataatattatcagtttctgatatgaaccaactatgatgtgtttatatcactattgggttatccatgtagcactggtggatcaccctatcactataaattaaccgttgacatagatttcagcgatgatgggatttctatccgacagtctggatgagtctggatgtgggtagatgtgagtctgaatgtgtgtagatgagcaagatgccaatgataaagtgcaattgttgaaaaaatagcttacctgatatggctctagccaaataagctctaattagtctaaacccctttagaacaaaagaatattttttgaatcctacatttttctatattttaaccaattcatgtgaaattcctatatcatccctatgttccaaagaggtccctagtgagtccctattccactataagagtatctctaactgtgatttaaatcagtgtcgtagtttaaaatatagggctcaaatcataaaaacagcttcaatagtgtcttattttatatttttcataaaaaaataatatggggcacaccataaagtgaccctaatatattacaccctaggttagcgtcatatccttgttttctacatcattctctacattttatttcccaataatacaatttacttcctaagatggatgatttagggcctaactgttgaagtacaatttgttttgttatgccttaacatgttaaattatgtattgttttaatttttgagggactattttgtggcacattgttggagatgctctttacctgtggcataaatcactgccacgtcttgaaatatagacttaatcattaaaaacaatcataacaatgccctattttacaaaattttatcaaaaaaatttagggcacaccataaagggacctaaatatactacatcccatattagtgtcatatctttattttgtacatcattcttaacattttttctaacaatataatttacttcctaaaatatataatttaaggttgaaactcaatttgtttttattgcattaaatatattaaatgatatagtattttaatttttggaaatgttattttttgagagacgagatactttagtaaaggtaacaatgagacgagaagtaatataaatatattgaaaattgacatgagttgtcatcggccgtcatgagctgaggatcctctccatttcgcacggagacggagtcccaggcacccagccaacctttttctactcttctggcagtctctgtctcccacacattctctctctaaactcccatgtcagcccgtctttatcgattaaggtcactttaccactagttaaagacgagcgccgcgtgcagacggagtggtagaaaaagctggatgtccatcgccttgcttccaggttttctccaaccatgtccaccaacacactgatctagtactaggactagtactagatcgatcctgcctgcctgcctgcttgtaacagcaacagcagcagaagtggcgcactgtgctttgcggtggcggaggctgcgagagaggaggaacttgcaagccgacttcc

SEQ ID NO: 2 - DNA, 60 bp, Artificial Sequence
  probe
gagaacaacctactcgtaccttgagtccattgtatgtagtaattaatgtgtttactacat

SEQ ID NO: 3 - DNA, 8884 bp, Zea maize
  misc_feature 7090, 7091, 7092, 7093, 7094, 7095, 7096, 7097, 7098,7099: n = A,T,C or G
aggatttctagagattcgtcataagacgcagtacccacgtggccatctcttagtcgtgtggaaaaggtgttgtgtgtgggaccaagacatgactcgccccagcctcgggcgactactctgggtccacaacagcgcctgaccccaccacatgggcgggtccggagccgccacgtgtccagagaaagtggtagaccccaaggcctcaacagtgagtccggacccccatgggagagtgtcggacccctggatatacagtccggacctccaagattggtccaggacctccacgtgtgcaagccagacccctagaatgggatctggacccccccccctcccccgtatggggtctaggacgcccacagtggggtcccaaggttccagggcagaacatactcgtgccttggtcaggacccaagcgggggtccgatgccaacatgtgtctagacctgatctggttatcacgtacactcctccccgctcaggcggagacccgatgctgccacgtggcatactacgcacggtattagccaacgggtggaacctggcatgacgcctctgggctacgcgcgccttcgcattcattacggataagacgtgcgcctgtccattccactgactggcagcgtgcccagtccacaatacgtgggccgcgcaattactcgcacgttactatatcgagggcaatcactcactattactcgtgcgggttactgcctatcaatgatgcatggactgcggtcatcatgactctcgctgattgctcatatacgttactaaggcggaggatccttgtgtactgtcagcatttactattcacataatgtattcctactattatgcttctgggcccacatgtcggggctcagcattcttgtatgtgcctcccttaaactataaaagggaaggcacacagcgttacaaggcacggacgctcaagttcgcacacaagctctcaaccacacagacacaggtttgacacatactcaatgctacatacacacagtggaggtagggtattacgctccgacgggcctgaaccactctaatccctcgtgtcctcgtgtgttcatcccaaatccacataacaggtaaaacgcttaggccccctcctcatcttaggattagggcgggtgcattccgtcacccggccggtgaatttctcccaccgacagatactttggcttgttaataaactagtgatataagaggctaattggcgagacattggtttataaggactccattatatagctatctagatattaacttaaattttcctttgtagttatatacgatgtattgtaacttgtgaaaaatgtttggtgcgtgtgagaaatgacaatgtggtattggacctatagtgacaattgacaatccactatcgtgtgatccggcactagtgtcatgagtataattgttgggttgttgtctaaactggtagtatcactcaggataccactaacggtttaagatgatatctggtaatgatatgtataaataatattatcagtttctgatatgaaccaactatgatgtgtttatatcactattgggttatccatgtagcactggtggatcaccctatcactataaattaaccgttgacatagatttcagcgatgatgggatttctatccgacagtctggatgagtctggatgtgggtagatgtgagtctgaatgtgtgtagatgagcaagatgccaatgataaagtgcaattggttgaaaaaatagcttacctgatatggctctagccaaataagctctaattagtctaaacccctttagaacaaaagaatattttttgaatcctacatttttctatattttaaccaattcatgtgaaattcctatatcatccctatgttccaaagaggtccctagtgagtccctattccactataagagtatctctaactgtgatttaaatcagtgtcgtagtttaaaatatagggctcaaatcataaaaacagcttcaatagtgtcttattttatatttttcataaaaaaataatatggggcacaccataaagtgaccctaatatattacaccctaggttagcgtcatatccttgttttctacatcattctctacattttatttcccaataatacaatttacttcctaagatggatgatttagggcctaactgttgaagtacaatttgttttgttatgccttaacatgttaaattatgtattgttttaatttttgagggactattttgtggcacattgttggagatgctctttacctgtggcataaatcactgccacgtcttgaaatatagacttaatcattaaaaacaatcataacaatgccctattttacaaaattttatcaaaaaaatttagggcacaccataaagggacctaaatatactacatcccatattagtgtcatatctttattttgtacatcattcttaacattttttctaacaatataatttacttcctaaaatatataatttaaggttgaaactcaatttgtttttattgcattaaatatattaaatgatatagtattttaatttttggaaatgttattttttgagagacgagatactttagtaaaggtaacaatgagacgagaagtaatataaatatattgaaaattgacatgagttgtcatcggccgtcatgagctgaggatcctctccatttcgcacggagacggagtcccaggcacccagccaacctttttctactcttctggcagtctctgtctcccacacattctctctctaaactcccatgtcagcccgtctttatcgattaaggtcactttaccactagttaaagacgagcgccgcgtgcagacggagtggtagaaaaagctggatgtccatcgccttgcttccaggttttctccaaccatgtccaccaacacactgatctagtactaggactagtactagatcgatcctgcctgcctgcctgcttgtaacagcaacagcagcagaagtggcgcactgtgctttgcggtggcggaggctgcgagagaggaggaacttgcaagccgacttccatggctgccgccttctccgccgctatcctcttccacctgctgctgctcctcttctcgccgtccgctgcgcagcctggtgagccgtcgctctcctgactccttccgagtcccaatccccattctcatgtccaaagattcgttttttttaatttcccctggttgttttgtttagggcggcaccgccctttctctacctagtttacgcatgcatactcatatagtcagtgtcatggggagaatgttccggcttctccagcagatttgcgagattgcaagaggaggaatgtgcatatccacactgaccccctgtttgattttgctcctttgcattccataaatgttagtgctagtaaatctaacctaacggagtaagcaagcaactactctgaattggctatctcccatcacgaagaatgggtttcacaaaattgccatcagactcctcggtcacaattataaccacaaggagtcaacgattacaatggcaaatagctaattatatccatttgtggaatgtgtgtgttgtactacatctaaacctcaccatctttaatttgcccccttcccatcctgctcctgcaggtttcattagcttggactgcgggggagctcgtgatcacacggatgccattgggatccagtggacctctgatgccagcttcgtctctggcggccaggccgcgcagctgctggtccagaatggcctgcagcagacccagcagcagctgaccaccgtgcgctacttccccgcggacaacaggaagcactgctacaccatgaacgtcaggaacaggacgcgctaccttgtcagggccactttcctctacggcaacttcgataacagcaacgtctacccgaagttcgatatctccctcggagcgtctccctggtccaccatcgtcgtcgacgacgccaccacccccgtcgttgaggaagccatcatcttggctgctgctcccacgctcagtgtatgtctctccaatgccagcacgggacagcccttcatctctactctggagcttcgacagtttaacggttcgctctactacaccactgacgagacacgcttctttcttggactgtctgcgaggataaattttggcgcaggaagcaatgattcagtgaggtactgatactgccacaccaccatgcgtactgatgccttttgcattgtacataggcttcaaatctggccacaattataaactaaaattgcatataaaaaatatataaacttagtatcctgtcctgtgtgtagaactatggctgcatgatttggtgattgttgttacattctctgtcctcgtcagctcaaagatccacagtcccatgtcactgttctgctgccacatatcaaaactctgatcaagcttcactttactgtcattactaagttataacaggaactacattttgtttcatgcaaacaaataacacaagagttttccttgtcttgttccacagatatcctgatgatccatttgatagaatctgggaatctgattctgtgaggagagcaaattaccttgttgatgttgctccagggactgaaagaatatcaactacaaaacccatatttgtcggtaccaacgaagaaccacctgaaaaggtcatgcaaacagcagtagttggccaggatgggtcattgaactaccgccttgatttggaaggtttcccagcaaatgcttggggagtctcgtattttgcagaaattgaagatttggcaccaaatgaaacgaggaaatttaagttagaggtccctggcatgccagcactcagtaaaccaactgttgatgtggaggagaatgctcaagggaaataccgtttgtatgaaccaggctacacgaatttgtcacttccgtttgttttctcctttgggttcaggaagacgaatgattcttcaaaggggcctattttgaatgccctggagatttacaaatatgtccaaattactatgggatcacaggatggtaagcaaattgtgcatggttctgctaccttttcttttttcctagctgcaacagcccattatgtttcagaataacaaatccactttggggtgtatgcaaaattcacagcaaatatcatggccagcatggtatcacgatatccacaggaaggttgggcacaagagggtggtgatccgtgcttaccagcatcatggtcctgggtgcaatgcagttcagaaacttctccaaggatattctcaatgtatgacaattgacaaccttgtcattctgcctttttttgccttaatgaattagtatttgacacctattaaaaaaatttccagcacattgtctgggaagaacattaccggaagtatcccggtggaactgacaaagctatcagggttggttgagctgtaaggactgaatactttatctttatcttgtctaagtatttggcttctattatctgatcattttacttccgttaatgcagaaggcttgatggtaattcattttctggccaaattcctgatttcagagaatgtgggaatttgcagtatatgtgagtttccagttattttgataatattttattcttaaatgattctactaatatactgtgctttgattctgaaatgattctactaatatatggtgcatttattcattctagtcaccttgagaacaatcagttaactggtgaattgccatcttctttgggtgacctacctaacctgaaagagttgtaagttaaacatccacgtgtacaccttgtcttaatctccacattttggaggccatagtataatattccttcaatttatttttcttcctgactttttttatttgtattcatttctttttttttctgctcaagaaaccaaaaggcaaattgatgtcagtgatttaggctttttcatatgattacatctatgtgaggatctttatggaaaaaagacctctcttattatgaatgaaaaaatttgcaggtatgttcaaaataacaagctgtctgggcaggttccaaaagcacttttcaagagaagcattattttgaagttagtaccctgtccttaaagcagattattaatttcagatcatgcttttgagtgaaacggcctctagctgggttggttagatgactagagtagcactttagtcctgagttcaactccccgtgagagcgaatttcaggcgaatttcagactagggttaaaaaatccttcgtctgtcccacatcagtggggcagagatttggcgggtttcttgacctgcgtgagaagctgtctgcctgtctcactgaatcaccaggttatgagttcgaaacagcctctccgcatttgcaaggggaaggcttgtttcggtttattccttacccatacctcactcatgtgggaggctccggcactgggtctgctctttttttatgtagaccaaatagctatgctgttcttagaagaatggtagcatgactcccatatgttctaatttgttacatgcctgcagcttctcaggtaacagtggccttcacatagtaagcaatggcattagccacaccataattgttatatgtctggtgattggagccgttgtcttactgggtgttgctattgggtgctatttcattacatgtaggagaaagaagaaatctcatgaaggtacaccttttagctcaatcgacatatctgatactatgctagagcatgcagtgattcttttgccatctccagacactgttgtcattgcagcagcaccagctaaaaaacttggttcatattttagtgaagtggctacagaatcagcacacagattttctttatctgaaattgaaaatgctactggcaaatttgagagaagaattgggtctggaggctttggcatagtatactatggaaagttggctgatgggagagagattgcagtcaaacttctcacgaatgactcgtatcagggaatcagagaattcttgaatgaggtctgttctcatactcgccacattagtaacctgaaatatggatcttagagtgtccgtgcatatatttatctggttaacctcttgtttcaggtgacattgctttccaagaatacatcatagacctcgaggcgnnnnnnnnnnaggtgacattgctttccagaatacatcatagacacctggttacattccttggttacagtcagcaagatggcaaaaacatactagtgtatgagttcatgcataatgggacactaaaagagcaccttcgtggtaagtccacatccaactgatggtaaatgtacatcaatgtcaaacagacagtaggtggaaagttattctcccacaagctaaatgttttacatcattagagattggatggaaggcgtgcatgcttgattaactgaactgaaacatgaaattatcagattctccttaatgatagacatatatctctgatttagctttgaattgtgattaattatgaatgatcaacctgggcaacaacatgaagtgttagatgacagcaccccagtgtcatagagaaaacgtatacaaatgagggtaaacctttggtggtgtttcgcaggagctgataatgaaaagataactagctggctgaagcgtcttgagattgcagaagattctgcgaaaggtttgtggtctgctacgtgttttttttttaggaaacaggaggggtagccccctcctaatggttaattaatatatattaataatagaagaaaaggatgtgtaattcaaggaacaccacaattggtgctctagccattattctataagagggaaatatatatatatattcttcgctctatggattctgaatgtgagctggtaaaaaaacaggtatagagtatctccacacgggatgctccccaacaatcatccatagagacctgaagagcagtaacattctcctagacaagaacatgagagcaaaagtggcagacttcggtctttcgaaacctgcagtggatgggtctcatgtgtcaagtatagttcgagggacagttggatacctggacccagagtaaggccaagcaaaatctgactgaggcgttttttttttctgtttcatcttgcgtctaaataaagaatcgattttttgtttgtgtgttctggttaggtactacatctcgcagcagctgacggagaagagcgacatctacagcttcggcgtgattttgctggagctcatctctgggcatgaacccatctcgaatgacaactttgggctcaactgccggaacattgttgcgtgggtaagaaagaagagtattcctgttggttctgctgaagcaaaagcgttaaataaataaataaataaatgcaggcccgatcgcacatcgagagcgggaacatccacgccatcattgatgagtcgttggacagaggctgctatgacctgcagtcggtgtggaagatcgcggaggtggccataatgtgtgtgaagcccaagggcgcccagaggcctcccatctcggaagtgctcaaggagatccaggacgccattgccatggagcgggtgctcgtgtccaattgcagcaacaggatgggttcaggtagcgtggagcagaatggaggagcatctttcgacgagctgctcatgcagccgggtctcagatgagatgagaacaacctactcgtaccttgagtccattgtatgtagtaattaatgtgtttactacattttttttgagaaacaaattaagtaatgctagcaaaggaaccagtatattattattattattattattattattattattattattattattattattattattattattattattattattattattattattattattattattattattattattattattattattattattattattattattattattattattattattattattattattataa

SEQ ID NO: 4 - DNA, 2817 bp, Zea maize
atggctgccgccttctccgccgctatcctcttccacctgctgctgctcctcttctcgccgtccgctgcgcagcctggtttcattagcttggactgcgggggagctcgtgatcacacggatgccattgggatccagtggacctctgatgccagcttcgtctctggcggccaggccgcgcagctgctggtccagaatggcctgcagcagacccagcagcagctgaccaccgtgcgctacttccccgcggacaacaggaagcactgctacaccatgaacgtcaggaacaggacgcgctaccttgtcagggccactttcctctacggcaacttcgataacagcaacgtctacccgaagttcgatatctccctcggagcgtctccctggtccaccatcgtcgtcgacgacgccaccacccccgtcgttgaggaagccatcatcttggctgctgctcccacgctcagtgtatgtctctccaatgccagcacgggacagcccttcatctctactctggagcttcgacagtttaacggttcgctctactacaccactgacgagacacgcttctttcttggactgtctgcgaggataaattttggcgcaggaagcaatgattcagtgagatatcctgatgatccatttgatagaatctgggaatctgattctgtgaggagagcaaattaccttgttgatgttgctccagggactgaaagaatatcaactacaaaacccatatttgtcggtaccaacgaagaaccacctgaaaaggtcatgcaaacagcagtagttggccaggatgggtcattgaactaccgccttgatttggaaggtttcccagcaaatgcttggggagtctcgtattttgcagaaattgaagatttggcaccaaatgaaacgaggaaatttaagttagaggtccctggcatgccagcactcagtaaaccaactgttgatgtggaggagaatgctcaagggaaataccgtttgtatgaaccaggctacacgaatttgtcacttccgtttgttttctcctttgggttcaggaagacgaatgattcttcaaaggggcctattttgaatgccctggagatttacaaatatgtccaaattactatgggatcacaggatgcaaatatcatggccagcatggtatcacgatatccacaggaaggttgggcacaagagggtggtgatccgtgcttaccagcatcatggtcctgggtgcaatgcagttcagaaacttctccaaggatattctcaatcacattgtctgggaagaacattaccggaagtatcccggtggaactgacaaagctatcagggttggttgagctgcttgatggtaattcattttctggccaaattcctgatttcagagaatgtgggaatttgcagtatattcaccttgagaacaatcagttaactggtgaattgccatcttctttgggtgacctacctaacctgaaagagttgtatgttcaaaataacaagctgtctgggcaggttccaaaagcacttttcaagagaagcattattttgaagttagtaccctgtaacagtggccttcacatagtaagcaatggcattagccacaccataattgttatatgtctggtgattggagccgttgtcttactgggtgttgctattgggtgctatttcattacatgtaggagaaagaagaaatctcatgaaggtacaccttttagctcaatcgacatatctgatactatgctagagcatgcagtgattcttttgccatctccagacactgttgtcattgcagcagcaccagctaaaaaacttggttcatattttagtgaagtggctacagaatcagcacacagattttctttatctgaaattgaaaatgctactggcaaatttgagagaagaattgggtctggaggctttggcatagtatactatggaaagttggctgatgggagagagattgcagtcaaacttctcacgaatgactcgtatcagggaatcagagaattcttgaatgaggtgacattgctttccagaatacatcatagacacctggttacattccttggttacagtcagcaagatggcaaaaacatactagtgtatgagttcatgcataatgggacactaaaagagcaccttcgtggagctgataatgaaaagataactagctggctgaagcgtcttgagattgcagaagattctgcgaaaggtatagagtatctccacacgggatgctccccaacaatcatccatagagacctgaagagcagtaacattctcctagacaagaacatgagagcaaaagtggcagacttcggtctttcgaaacctgcagtggatgggtctcatgtgtcaagtatagttcgagggacagttggatacctggacccagagtactacatctcgcagcagctgacggagaagagcgacatctacagcttcggcgtgattttgctggagctcatctctgggcatgaacccatctcgaatgacaactttgggctcaactgccggaacattgttgcgtgggcccgatcgcacatcgagagcgggaacatccacgccatcattgatgagtcgttggacagaggctgctatgacctgcagtcggtgtggaagatcgcggaggtggccataatgtgtgtgaagcccaagggcgcccagaggcctcccatctcggaagtgctcaaggagatccaggacgccattgccatggagcgggtgctcgtgtccaattgcagcaacaggatgggttcaggtagcgtggagcagaatggaggagcatctttcgacgagctgctcatgcagccgggtctcagatga

SEQ ID NO: 5 - protein, 938 aa, Zea mays
MAAAFSAAILFHLLLLLFSPSAAQPGFISLDCGGARDHTDAIGIQWTSDASFVSGGQAAQLLVQNGLQQTQQQLTTVRYFPADNRKHCYTMNVRNRTRYLVRATFLYGNFDNSNVYPKFDISLGASPWSTIVVDDATTPVVEEAIILAAAPTLSVCLSNASTGQPFISTLELRQFNGSLYYTTDETRFFLGLSARINFGAGSNDSVRYPDDPFDRIWESDSVRRANYLVDVAPGTERISTTKPIFVGTNEEPPEKVMQTAVVGQDGSLNYRLDLEGFPANAWGVSYFAEIEDLAPNETRKFKLEVPGMPALSKPTVDVEENAQGKYRLYEPGYTNLSLPFVFSFGFRKTNDSSKGPILNALEIYKYVQITMGSQDANIMASMVSRYPQEGWAQEGGDPCLPASWSWVQCSSETSPRIFSITLSGKNITGSIPVELTKLSGLVELLDGNSFSGQIPDFRECGNLQYIHLENNQLTGELPSSLGDLPNLKELYVQNNKLSGQVPKALFKRSIILKLVPCNSGLHIVSNGISHTIIVICLVIGAVVLLGVAIGCYFITCRRKKKSHEGTPFSSIDISDTMLEHAVILLPSPDTVVIAAAPAKKLGSYFSEVATESAHRFSLSEIENATGKFERRIGSGGFGIVYYGKLADGREIAVKLLTNDSYQGIREFLNEVTLLSRIHHRHLVTFLGYSQQDGKNILVYEFMHNGTLKEHLRGADNEKITSWLKRLEIAEDSAKGIEYLHTGCSPTIIHRDLKSSNILLDKNMRAKVADFGLSKPAVDGSHVSSIVRGTVGYLDPEYYISQQLTEKSDIYSFGVILLELISGHEPISNDNFGLNCRNIVAWARSHIESGNIHAIIDESLDRGCYDLQSVWKIAEVAIMCVKPKGAQRPPISEVLKEIQDAIAMERVLVSNCSNRMGSGSVEQNGGASFDELLMQPGLR

SEQ ID NO: 6 - protein, 903 aa, Phaseolus vulgaris
MWITFYVAVLAVLLLQAHAQPGFISIDCGAEAGVSYTERSLGINYVSDANFINTGERRTIASEEISRNNQQQQLWRLRSFPEGKRNCYKINVTSGSNYLIRTTFLYGNYDGRNKLPMFDLLLGANLWSTVTIDDASSGQSNEIIHVPSLDFVQICLVNTGSGTPFITAIEFRTLKNDTYVTESGSLQSSLRWDLGSNISYRYPTDVYDRFWNPQDNKDWTNLSASIPDDSLDQGDYQPGASNMRTAVTPANASAPLVISWEPKDETDEFYVYMHFTEIQELTTNQTRQFDIMRNGELWIPNFSPRYLVVDTLNTSSASAVNGKVITYSLVRTGNSTLPPIISAIEIYRVIDLQKPETLQADVDAITSIDSVYGVKRDWQGDPCAPVAYLWNGLNCSYHGIEFPRITALNLSSSGLSGKIDPSISKLNMLEKLDLSNNNLHDEVPDFLSQLQHLKILHLEKNNLSGSIPSALVEKSKEGSLTLSLGQNPHICEHGQCIDHRNNIVIPLVASICFFLILLVTVTAILWILRRRRKSKASMVEKDQSEISEQHTKQEGSLQQSKKQICSHSDICKITNNFNTIVGKGGFGTVYLGYIYDTPVAVKILSPSSFRGYEQFQAEVTLLLRVHHKNLTSLIGYCDEGSNKSLIYEYMANGNLLEHLSGTHSKSKFLSWEDRLRIAVDAALGKKANFRLCVLTVLLMWHLHKSNIGLEYLQNGCKPPIIHRDVKSSNILLNEHFQAKLSDFGLSKIIPDDGASHLTVVAGTPGYLDPDYYTNNRLTEKSDVYSFGVVLLEIITGQLVIARNEERSHITEWVRSLVAIGDIKAIVDSRLEGDFDINSAWKAVEIAMACVSLRPNQRPIMSVVVFELKETLVTELARTKPNSSAESIEPVNLDFSAQLIPLAR

SEQ ID NO: 7 - protein, 426 aa, Artificial Sequence
  consensus
LLLLAAQPGFISIDCGGAIGINWSDAFIGNNQQQLLRFPKKCYINVSYLIRTFLYGNFDNPFDILGAWSTIIDDASSEILALICLNSGPFISIERYTSLAIFGSNSRYPDFDRWKLPPMTAVASIEWHFEIDLNTRFIGLPNVSFKTSSPIIAIEIYKIISAIAKGDPCPAWWPRIAILSISGIISKLLILNNNLELPLLLKLHLNLSGIPALKSILLCGIIVIGILLLVWIRRKKLDIAKGSLSSDITFIGGGFGVYGIDIAVKILSSFGFEVTLLRIHHKLSIGYLIYEFMNGLEHLGKSWRLIADAAGIEYLGCPIIHRDLKSSNILLAKLADFGLSKDSHLSSIVGTGYLDPDYYNLTEKSDIYSFGVILLEIISGIAIIWRFIGIAIIDLDFDINSWKEIAICVKQRPISVLEIDIESVAL